(19) United States
(12) Patent Application Publication
(10) Pub. No.: US 2024/0158860 A1
(43) Pub. Date: May 16, 2024

(54) CIRCULATING miRNA MARKERS FOR DETECTION OF RADIATION EXPOSURE

(71) Applicants: THE GOVERNMENT OF THE UNITED STATES, AS REPRESENTED BY THE SECRETARY OF THE AR, Fort Detrick, MD (US); THE UNITED STATES OF AMERICA, AS REPRESENTED BY THE SECRETARY, DEPARTMENT OF HE, Bethesda, MD (US)

(72) Inventors: Nabarun M. CHAKRABORTY, Rockville, MD (US); Sanchita P. Ghosh, North Potomac, MD (US); Aarti Gautam, North Potomac, MD (US); Raina Kumar, Ashburn, VA (US); Rasha Hammamieh, Germanton, MD (US)

(21) Appl. No.: 18/507,575

(22) Filed: Nov. 13, 2023

Related U.S. Application Data
(60) Provisional application No. 63/424,554, filed on Nov. 11, 2022.

(51) Int. Cl. C12Q 1/6883

(52) U.S. Cl. CPC C12Q 1/6883; C12Q 2600/118; C12Q 2600/158; C12Q 2600/178

(57) ABSTRACT

Early diagnosis of lethal radiation is imperative, since the intervention time windows for lethally irradiated subjects are considerably short. Hence, ideal diagnostic candidates of radiation should be easily accessible, enable to inform the stress history and triage subjects unbiasedly in a time-efficient manner. Taken together, small molecules such as metabolites and microRNAs (miRNAs) from plasma are legitimate biomarker candidate for lethal radiation. Herein is described a TBI (total body irradiation) model involving minipigs exposed to radiation. Markers were observed at early, intermediate and late time periods post-radiation, and were compared to non-exposed, non-radiated miRNA's. A method and a kit for detecting time-dependent ionizing radiation exposure is described in embodiments herein.

### BACKGROUND

Radiological casualties are events that must be identified and managed, and require immediate intervention by medical personnel. Catastrophic events such as terrorist attacks, or nuclear power plant failures may cause large scale radiation exposure which may rapidly impact individuals who are exposed. Any such exposure must be expeditiously diagnosed and treated. Certain protocols are established for nuclear exposure management and assessment of those who have been affected and require immediate medical treatment. Certain factors must be observed to differentiate those in need of treatment from those who are not in need of treatment for exposure. Radiation exposure may not immediately result in visible damage to an individual, and therefore, may be difficult to diagnose. Consequently, methods for detecting radiation exposure has occurred, and for determining length of time from radiation exposure are critical for treatment.

### SUMMARY

According to one embodiment, provided is a method for detecting exposure to ionizing radiation in a subject. The method involves assaying a biological sample to detect the presence of one or more biomarkers that are downregulated or upregulated relative to a normal non-irradiated biological sample to determine if radiation exposure has occurred, and, optionally, the approximate time lapsed since the radiation exposure. The biological sample is, optionally, a blood sample, serum or plasma, from a subject at risk of exposure to ionizing radiation. Conditions indicative of radiation exposure include one or more of the following:
i. downregulation of one or more Group A miRNAs relative to normal non-irradiated serum or plasma sample, wherein Group A miRNAs comprise miR-432-5p, miR-221-5p, miR-92b-5p, miR-331-3p, miR-424-5p, miR-376a-3p and/or miR-374b-5p; and/or
ii. upregulation of one or more Group B1 miRNAsrelative to normal non-irradiated serum or plasma sample, wherein Group B1 miRNAs miR-218-5p, miR-96-5p, miR-671-5p, miR-424-3p, miR-490-3p, miR-296-5p, miR-769-3p, miR-27b-5p, miR-190b, miR-374b-3p, miR-193a-3p, miR-202-3p, miR-885-3p and/or miR-217; and/or
iii. downregulation of Group B2 miRNAs relative to normal non-irradiated serum or plasma sample, wherein Group B2 miRNAs comprise miR-532-3p, miR-885-5p, miR-545-5p, miR-1296-5p, miR-133b, miR-206, miR-432-5p, miR-221-5p, miR-92b-5p, miR-331-3p, miR-424-5p, miR-30c-1-3p, miR-676-3p, miR-542-3p, miR-542-5p, miR-184, miR-193a-5p, miR-376a-3p, miR-374b-5p, miR-139-5p, miR-545-3p, miR-133a-3p, miR-133a-5p and/or miR-1306-3p; and/or
iv. upregulation of one or more Group C1 miRNAsrelative to normal non-irradiated serum or plasma sample, wherein Group C1 miRNAs comprise, miR-218-5p, miR-190b, miR-339-3p, miR-429 and/or miR-676-3p; and/or
v. downregulation of one or more Group C2 miRNAs relative to normal non-irradiated serum or plasma sample, wherein Group C2 miRNAs comprise let-7d-5p, miR-1306-5p, miR-139-3p, miR-140-5p, miR-142-3p, miR-142-5p, miR-145-3p, miR-148a-5p, miR-148a-5p, miR-148b-5p, miR-155-5p, miR-17-3p, miR-17-5p, miR-181d-5p, miR-199a-5p, miR-199b-5p, miR-221-3p, miR-22-3p, miR-22-5p, miR-24-1-5p, miR-24-3p, miR-27b-3p, miR-28-3p, miR-28-5p, miR-296-3p, miR-30a-3p, miR-30b-5p, miR-30c-5p, miR-30e-3p, miR-326, miR-331-5p, miR-345-3p, miR-361-3p, miR-361-5p, miR-374a-3p, miR-374a-5p, miR-425-3p, miR-425-5p, mir-450b-5p, miR-455-3p, miR-455-5p, miR-532-5p, miR-671-3p, miR-6782-3p, miR-769-5p, miR-1296-5p, miR-133b, miR-542-5p, miR-184, miR-193a-5p, miR-545-3p, miR-133a-3p, miR-432-5p, miR-221-5p, miR-92b-5p, miR-331-3p, miR-424-5p, miR-30c-1-3p, miR-542-3p, miR-376a-3p, miR-374b-5p, miR-139-5p, miR-545-3p, miR-133a-3p, miR-133a-5p and/or miR-1306-3p; and/or
vi. upregulation of one or more Group D1 miRNAsrelative to normal non-irradiated serum or plasma sample, wherein Group D1 miRNAs comprise miR-218-5p, miR-296-5p, miR-126-5p, miR-424-3p, miR-143-3p, miR-345-3p, miR-708-3p and/or miR-708-5p; and/or
vii. downregulation of one or more Group D2 miRNAsrelative to normal non-irradiated serum or plasma sample, wherein Group D2 miRNAs comprise miR-181d-5p, miR-24-1-5p, miR-331-5p, miR-432-5p, miR-221-5p, miR-92b-5p, miR-331-3p, miR-424-5p, miR-376a-3p, miR-374b-5p and/or miR-1306-3p,
   The method optionally involves administering a radiation injury therapy to the subject if any of the above conditions i-vii are present.

In a specific examples, upregulation of Group B1 miRNA's or downregulation of Group B2 miRNA's, or a combination thereof, is an early marker indicative of approximately 0-11 months post-exposure to ionizing radiation; upregulation of Group C1 miRNA's or downregulation of Group C2 miRNA's, or a combination thereof, is an intermediate marker indicative of approximately 5-9 months post-exposure to ionizing radiation; and upregulation of Group D1 miRNA's or downregulation of Group D2 miRNA's, or a combination thereof, is a late marker indicative of approximately at least 12 months post-exposure to ionizing radiation.

In the above method, detecting may involve subjecting the biological sample to an amplification reaction using primers directed to the one or more biomarkers. Alternatively, detecting may involve subjecting the biological sample to hybridization-based methods, NanoString™ analysis, Northern Blot analysis, branched DNA (bDNA) signal amplification, in situ hybridization, sequencing-based methods, ribonuclease protection assay (RPA) and/or mass spectroscopy.

According to another embodiment, provided is a radiation exposure detection kit that includes one or more reagents for detecting one or more biomarkers. The one or more biomarkers include:
a first time-independent marker for detecting one or more of the following miRNAs in a blood serum or plasma sample, comprising: miR-432-5p, miR-221-5p, miR-92b-5p, miR-331-3p, miR-424-5p, miR-376a-3p and miR-374b-5p;
a second, early detection biomarker for detecting upregulation of one or more of Group B1 miRNA's or downregulation of one or more of Group B2 miRNA's, or a combination thereof;
a third, intermediate detection biomarker for detecting upregulation of one or more of Group C1 miRNA's or downregulation of one or more of Group C2 miRNA's, or a combination thereof; and/or
a fourth, late detection biomarker for detecting upregulation of one or more of Group D1 miRNA's or downregulation of one or more of Group D2 miRNA's, or a combination thereof;
   The allows for determination of whether a) exposure to ionizing radiation has occurred, and/or b) the approximate time since exposure. In a specific example, the one or more reagents includes primers directed to the one or more biomarkers and/or one or more reagents comprises probes directed to the one or more biomarkers. The probes may also include a detectable label.

The kit may be used in a method for detecting exposure to ionizing radiation in a subject. This method involves using the kit to assay a biological sample for the presence of biomarkers that are downregulated or upregulated relative to a normal non-irradiated biological sample, wherein the biological sample is, optionally, a blood sample comprising serum or plasma, from a subject at risk of exposure to ionizing radiation to determine if radiation exposure has occurred, and, optionally, the approximate time lapsed since the radiation exposure. The one or more reagents of the kit may include primers directed to the one or more biomarker and probes directed to the one or more biomarkers. The method may further involve subjecting the biological sample to an amplification reaction. Examples of useful amplification reactions include Polymerase Chain Reaction (PCR), Real-Time Polymerase Chain Reaction (RT-PCR), Quantitative Polymerase Chain Reaction (qPCR), or rolling circle amplification.

According to another embodiment, disclosed is a method of determining biomarkers indicative of radiation exposure. The method involves a) irradiating a test animal, to produce an irradiated animal; b) collecting a test sample from the irradiated animal; c) analyzing the test sample for differentially expressed miRNAs relative to a control animal; and d) identifying differentially expressed miRNAs that are mapped to human miRNAs. Identifying may involve identifying differentially expressed miRNAs in the test animal that are conserved with respect to human miRNAs or identifying differentially expressed miRNAs functionally similar to human miRNAs. Identifying conserved with respect to human miRNAs involves pooling differentially expressed miRNAs, subjecting pooled differentially expressed miRNAs to a multiple sequence alignment tool, and conducting pairwise sequence alignment between test animal miRNA and human genome at predetermined loci position. Examples of a multiple sequence alignment tool include CLUSTL4, and conducting involves applying parameters for alignment comprising a k-tuple word size 1 and window size 5 with top diagonals of 5 with percent method, and a gap penalty of 10 and penalty of extension 0.1 with BLOSUM scoring weight matrix. In another specific example, identifying differentially expressed miRNAs functionally similar to human miRNAs involves pooling differentially expressed miRNAs, subjecting pooled differentially expressed miRNAs to a multiple sequence alignment tool, and conducting pairwise sequence alignment between test animal miRNA and human genome at predetermined loci position, wherein conducting pairwise sequence alignment comprises subjecting differentially expressed miRNAs to miRDeep2 core algorithm in miRbase.

These and other embodiments are described, illustrated, supported and claimed below.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1. (A) is a schematic of the study design. The 4 months old male sus surcofa were exposed to lethal total body irradiation (TBI). The terminal plasma samples were collected at two time points before the irradiation, namely 7 days (C7) and 1 day (C1) pre-TBI. Six hours after TBI, the first terminal plasma samples were collected and is marked as 0d. Subsequently, terminal plasma samples were collected at seven time points, namely 1 day (1d), 3 day (3d), 7 day (7d), 10 day (10d), 14 day (14d), 17 day (17d) and 20 day (20d) post-TBI, respectively. Each time points were coded in the flow diagram and same codes were used in the next figure demonstrating the Principal Component Analysis (PCA). Plasma samples were used for microRNA (miRNA) assays.

FIG. 1 (B) is a PCA plot of all plasma miRNAs expressed in individual time points. PC1 and PC2 explained 29.3% and 11.2% of total variance, respectively. Seven days and one day pre-TBI time points were named as −7d and −1d, respectively and were labeled as wheel shapes colored by yellow and white, respectively. Post-TBI time points were divided into four phases based on their degree of separation from the pre-TBI samples. The early time points that essentially clustered together with baselines were named Early phase. This time cluster included 0d, 1d, 3d and 7d post-TBI time points marked as the circles colored by green, orange, gray and blue, respectively. Ten day (10d) and 14d post-TBI time points were marginally separated from this baseline and Early phase conglomerate, which were named as Mid phase and labeled by squares colored by green and blue, respectively. Late phase or 17d post-TBI samples were clustered furthest from the conglomerate made by the baseline and Early phase. Blue colored ‘X’s labeled the Late phase. A light blue colored triangle marked the 20d post-TBI time points, which were named as Delayed regression phase to acknowledge the fact that these samples clustered back to the conglomerate of baseline and Early phase.

FIG. 1(C) is a flow chart depicting an in silico model to identify the clinically actionable miRNAs. The miRNA reads were mapped to both sus sarcofa and human genome in parallel. The null hypothesis of this model was that these two independent read-mapping pipelines should generate a completely overlapping panel of miRNAs, since the sequences of miRNAs are believed to be conserved across the phylogenetic tree. Contrasting to the pre-TBI baseline, the differentially expressed miRNAs at individual post-TBI temporal phases were identified for these two parallel pipelines. The miRNA reads mapped to sus sarcofa delivered 212 ssc-miRNAs, which were differentially expressed in at least one of the four temporal phases, namely Early, Mid, Late and Delayed regression. Sequence alignment of these ssc-miRNAs to human genome delivered a re-annotated panel of 212 hsa-miRNAs. The parallel pipeline mapping the miRNA reads directly to human genome found 110 hsa-miRNAs, which were differentially expressed in at least one of the four temporal phases. There were 92 miRNAs (40% of combined list) conserved between these two sets of hsa-miRNAs. FIG. 4 depicted all of these 92 miRNAs and Table 2 listed a subset of this list, which showed strong translational potential to be a putative panel of early markers of lethal radiation.

FIG. 1(D) is a Venn diagram of ssc-miRNAs (minipig miRNA). The differentially expressed ssc-miRNAs of all four temporal phases were compared among themselves and to the 92 miRNAs that emerged conserved between the two parallel pipeline explained in FIG. 1C.

FIG. 2(A) is a chart showing percentage share of the network categories across the temporal phases. The stacked bar plots represent the six dominating categories of networks. The group of networks labeled ‘others’ were linked to cell movement, cell cycle, neurological illness, developmental disorder etc.

FIG. 2(B) is a graph showing the top five most significantly perturbed networks linked to Early time point. There were 37 networks that were consistently enriched across all four temporal phases, i.e. from Early to LR. Likewise, there were 41 and 4 networks that were consistently enriched in two other chronological combinations of temporal phases, i.e. from Early to Late and from Early to Mid, respectively. These networks were arranged in ascending order of their hypergeometric p values and five top ranked networks were selected from the two combinations of temporal phases, i.e. from Early to LR, and from Early to Late, respectively. All four networks from the third combination, i.e. from Early to Mid were selected. These networks emerged significantly enriched in at least two chronologically consecutive phases starting from the Early phase. The bar graphs were plotted against the top x-axis represents −log(p-value) and the solid black circles plotted against the bottom x-axis represents the number of miRNAs enriching individual networks.

FIG. 2(C) is a map providing a network linked to gene silencing. A regulatory network linked to the noncoding RNA residing in the homeobox C (HOXC) locus, termed HOTAIR emerged significantly altered within this framework of gene silencing network. In the network, the rectangular and oval nodes represent biofucntions and miRNAs, respectively. The four segments of the bar at the bottom of each miRNAs documented the regulations in four temporal phases arranged chronologically from left to right. The red, green and yellow colored segments represented up-, down and unchanged regulations in comparison to the pre-TBI baseline. The edges represent the relationship between two nodes; the arrow-headed edges represent activation and the straight lined edges represent association between two nodes.

FIG. 3 provides the hierarchical clustering of differentially expressed (DE) miRNAs conserved between two pipelines explained in FIG. 1C. Ninety two DE miRNAs were hierarchically clustered following Euclidian algorithm and Ward's linkage rule. The consistently regulated miRNAs across in at least two temporal phases starting from Early-phase were of particular interest; the corresponding nodes were boxed and the miRNAs were listed in Table 1. The color code corresponding to the log (fold change) was at the right bottom.

FIG. 4 shows a Venn diagram of the functions linked to the miRNAs differentially expressed (DE) in the post-TBI four temporal phases.

FIG. 5 provides a hierarchical clustering of differentially expressed (DE) miRNAs linked to cancer. One hundred and three DE miRNAs were hierarchically clustered following Euclidian algorithm and Ward's linkage rule. The color code corresponding to the log(fold change) was at the right bottom.

### DETAILED DESCRIPTION

### Overview

The need to build a comprehensive knowledge about the nuclear radiation exposure is more pertinent than ever given the permeation of ionizing radiation in our society's framework. Not only has the threat of global nuclear events become more imminent in recent years¹, but the escalated handling of nuclear radiation in industrial and medical sectors also pose risks for accidental radiation exposure. Exposure to ionizing radiation causes dose-dependent health effects and are characterized as Acute Radiation Syndrome (ARS). Adverse effects in humans can be induced after exposure to as little as 2 Gy^{2,3} of radiation resulting from damage to proliferating cells in the most radio-sensitive system, namely the hematopoietic structure. Hematopoietic ARS (H-ARS) is characterized by depletion of circulating blood cells, induction of widespread inflammation and endothelial dysfunction⁴⁻⁶ which lead to potentially life-threatening infection and hemorrhaging^{4,7}. Longitudinal and dose-dependent evaluation of H-ARS imperative to develop precise intervention strategy.

Development of biomarkers for fast and accurate dose assessment post radiation exposure is critical. Each individual's response to an exposure varies depending on many confounding factors, such as immune status, age and genetics. These factors will ultimately determine a person's response to exposure, and in some cases, victims may not immediately exhibit visible signs of radiation damage. Therefore, physical dosimetry alone, and the available protein markers such as cytokines, have limitations to accurately estimate the dose and response of an individual.

Since, nearly all of the radiation injured patients have preexisting confounders, such as cancer, animal models are possibly the best source to study the exclusive impacts of radiation. Hence the animal models are crucial in the advancement of understanding radiation effects as well as studying countermeasures and treatments for these effects². Traditionally, the mouse models are the most popular in radiation research field due to its availability, cost, size, lifespan, and homology to human genotype and physiology^{2,8,9}. However, significant differences between mouse and human in context of their skin textures and surface areas limits the applicability of mouse model in studying total body irradiation (TBI)². The logistic burdens and mounting ethical concerns of using non-human primates in the radiation study somewhat outweighs its obvious applicability to radiation research^{2,9-12}.

Taking all these aspects in consideration, the Gottingen minipig (GMP) emerges as the most scientifically meaningful and logistically manageable animal model for advancing the knowledge of ARS pathophysiology. In general, pigs have immune systems similar to the immune system of humans¹³, similar metabolism¹⁴, and similar cardiopulmonary systems¹⁵. Swine are highly regarded as a translation model for ischemic stroke, diabetes, obesity, traumatic brain injury, coronary artery disease, and infarction¹⁵⁻²². With a proportionate ratios between body weight and skin area, comparable skin surface areas (0.5-1.0 m² of minipig vs. 1.8-2.0 m² in human), and similar skin textures, minipig emerge as a legitimate TBI model²³. Furthermore, minipig genome scores 84% homology with human, which is higher than that of homology between mouse and human²⁴. Recently, the FDA approved the transplantation of porcine skin graft to humans and transplantation of heart valves from porcine origin has been common practice for the last 60 years. The close similarity between human and porcine anatomy and physiology, availability, and extensive use in biomedical research make this model well-suited for biodosimetric applications and radiation countermeasure testing^{2,9}. We have previously characterized the GMP as a model of H-ARS showing the GMP displays closely aligned ARS development and symptoms to those documented in non-human primates, canines, and humans²⁵⁻²⁸. Specifically, H-ARS in the GMP shows prodromal, latent, manifest, and morbidity or recovery phases of the disease as well as severe decline in peripheral blood cell counts, inflammation, hemorrhage, and organ dysfunction²⁵⁻²⁸. Additionally, we have characterized the use of the GMP H-ARS model for countermeasure testing^{27,29,30}. Tissue-specific patterns of radiation-induced gene expression changes with regards to outcome have also been established in our GMP H-ARS model³¹.

Assessing the pathophysiology of radiation exposure in this model is dependent on identifying and studying biomarkers for detection of exposure and prognosis which may allow for early decision on medical management, triggers for treatment, and triage. Circulating microRNAs (miRNAs) are promising biomarkers of radiation damage³²⁻³⁷. Malachowska et al. reviewed the irradiation-inked miRNA profiles reported by 467 studies and suggested dose-dependent putative miRNA profiles for radiation exposure³². One of the miRNAs identified by Malachowska et al.³², miR-30c, which is associated with atherosclerosis and heart disease³², has been show to play a role in radiation response³⁶ with countermeasure, delta-tocotrienol, mitigating the radiation-induction of this miRNA in a mouse model and a CD34+ human cell line³⁸. In a mouse ARS model, up-regulation of miR-27b, miR-126, and p53 target, miR-34a³⁵ was found to be modulated by administration of a prophylactic radiation countermeasure, vitamin E isomer, gamma-tocotrienol. Of seven miRNAs significantly changed 24 hours post-irradiation in rhesus macaques, miR-133b, miR-215, and miR-375 accurately distinguished exposure and miR-30a and miR-126 accurately delineated based on survival outcome^{34,39}. In their review, Singh and Pollard highlight the lack of miRNA studies in animal models outside of NHP and mice³⁴. The field of miRNA research is still in its infancy and needs to be validated across multiple conditions₃₄.

Changes in metabolomics following exposure to radiation is an emerging field⁴⁰, Radiation-induced metabolic changes can be observed before the clinical onset of systems and accordingly could be used to augment current methods of triage for exposed individuals^{40,41}. Metabolic profiles have been studied in mouse and rat biospecimens such as plasma urine, and tissues⁴²⁻⁴⁶ and in previous reports, we have demonstrated radiation-induced alterations of metabolic profiles^{33,41} in a mouse model of ARS. Metabolic profiles in radiotherapy patients have also been studied^{47,48}, and increased levels of biomarkers involved in energy metabolism were observed in these patients⁴⁸. As with miRNA profiling, metabolite profiling following ionizing radiation exposure is an understudied field. A recent study in rhesus macaques has demonstrated that exposure to ionizing radiation leads to alterations to insulin response in tissues⁴⁹. Additional studies have demonstrated alterations in amino acid distribution^{50,51} and levels of proteins involved in protein synthesis⁵² following exposure to ionizing radiation.

Data provided herein supports embodiments that fill the gap in both of these knowledge bases using a GMP model of H-ARS. Presented herein is miRNA and metabolic profiling data from three animals that did not survive total body irradiation at 2.2 Gy (a dose that produces 70% lethality in the 45 day period post-irradiation). To assess the temporal changes in these profiles in response to radiation, blood collected at various times post-irradiation and categorized into four phases, Early (days 0-7), Mid (days 10-14), Late (day 17) and Late regression (LR) of H-ARS (day 20), with baselines established prior to exposure. In view of the above, it is essential to develop methods for detecting radiation exposure and to approximate the time since exposure, as described herein.

As aforementioned, in some embodiments, where a subject is determined by the methods described herein to have been exposed to high doses of radiation, for example, enough to result in acute radiation syndrome (ARS), also disclosed are methods of treating such subjects for radiation poisoning.

ARS, also known as radiation poisoning, radiation sickness, or radiation toxicity, is a constellation of health effects which present within 24 hours of exposure to high amounts of ionizing radiation. The radiation causes cellular degradation due to damage to DNA and other key molecular structures within the cells in various tissues; this destruction, particularly as it affects ability of cells to divide normally, in turn causes the symptoms. The symptoms can begin within one or two hours and may last for several months. The terms refer to acute medical problems rather than ones that develop after a prolonged period. The onset and type of symptoms depends on the radiation exposure. Relatively smaller doses result in gastrointestinal effects such as nausea and vomiting and symptoms related to falling blood counts such as infection and bleeding. Relatively larger doses can result in neurological effects and rapid death.

Similar symptoms may appear months to years after exposure as chronic radiation syndrome when the dose rate is too low to cause the acute form or as delayed or late effects of the acute exposure. Radiation exposure can also increase the probability of developing some other diseases, mainly different types of cancers. These diseases are sometimes referred to as radiation sickness, but they are never included in the term acute radiation syndrome.

Also disclosed herein in embodiments are methods and a kit for measuring exposure of a mammalian subject to ionizing radiation. The methods generally involve determining in a cell-free biological sample (e.g., serum or plasma) from the subject the levels of at least one radiation-sensitive miRNA whose blood levels are radiation dose- and time-dependent.

Various miRNA's have been identified in irradiated plasma or serum samples as either downregulated or upregulated following radiation exposure as compared to normal non-irradiated plasma or serum samples, either immediately, or after a duration of time following exposure as early, intermediate, or late markers based on time post irradiation.

Examples of radiation-sensitive miRNA disclosed herein are included below.

The embodiments disclosed herein are based on the identification of a longitudinal set of circulating microRNAs (MiRNAs) from minipig blood exposed to lethal radiation. Differential expression analysis and subsequent data dimension reduction approach identified the miRNAs of the following time-dependent and mutually exclusive categories.
Group A includes miRNAs that were consistently regulated across entire time elapsed since irradiation (time independent miRNA markers);
Group B includes miRNAs that were exclusively expressed immediately after irradiation (early markers);
Group C includes miRNAs that were exclusively expressed at the intermediate times after irradiation (intermediate markers); and
Group D includes miRNAs that were differentially expressed at late time points since irradiation (delayed markers).

Subsequently, it was determined which of the foregoing minipig miRNAs are sequentially conserved between human and minipig. A set of miRNAs was determined that can be detected and quantified using convention molecular detection techniques, such as PCR, NanoString, customized microarray or targeted sequencer. Detection of the miRNAs (with appropriate expression level) of “Group A” provides an indication of the occurrence of irradiation. In conjunction, the detection of the miRNAs (with appropriate expression level) of “Group B, C and D” provides an indication about the time elapsed since radiation.

Embodiments described herein enable the following:
1. Diagnosis of the exposure to lethal radiation
2. Determination of the time elapsed since the lethal radiation exposure
3. Early diagnosis of the exposure to lethal radiation
4. Late determination of the occurrence of lethal radiation
5. A rapid and bench side assay using minimally invasive biomatrix, such as serum or plasma

### Definitions

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that the throughout the application, data are provided in a number of different formats, and that these data represent endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “radiation injury” refers to an injury or damage that is caused by exposure to ionizing radiation. Radiation injury includes but is not limited to radiation poisoning, radiation sickness, acute radiation syndrome or chronic radiation syndrome.

As used herein, the term “detecting” refers to observing one or more indicators of the presence of a biomarker in the sample. Any method known in the art for detecting a particular detectable moiety can be used for detection. Exemplary detection methods include, but are not limited to, spectroscopic, photochemical, biochemical, immunochemical, electrical, optical or chemical methods.

The term “biomarker value” refers to a value measured or derived for at least one corresponding biomarker of the biological subject and which is typically at least partially indicative of a concentration of the biomarker in a sample taken from the subject. Thus, the biomarker values could be measured biomarker values, which are values of biomarkers measured for the subject, or alternatively could be derived biomarker values, which are values that have been derived from one or more measured biomarker values, for example by applying a function to the one or more measured biomarker values.

Biomarker values can be of any appropriate form depending on the manner in which the values are determined. For example, the biomarker values could be determined using high-throughput technologies such as mass spectrometry, sequencing platforms, array and hybridization platforms, immunoassays, flow cytometry, or any combination of such technologies and in one preferred example, the biomarker values relate to a level of activity or abundance of an expression product or other measurable molecule, quantified using a technique such as PCR, sequencing or the like. In this case, the biomarker values can be in the form of amplification amounts, or cycle times, which are a logarithmic representation of the concentration of the biomarker within a sample, as will be appreciated by persons skilled in the art and as will be described in more detail below.

The term “labeling probe” generally, according to various embodiments, refers to a molecule used in an amplification reaction, typically for quantitative or qPCR analysis, as well as end-point analysis. Such labeling probes may be used to monitor the amplification of the target polynucleotide. In some embodiments, oligonucleotide labeling probes present in an amplification reaction are suitable for monitoring the amount of amplicon(s) produced as a function of time. Such oligonucleotide labeling probes include, but are not limited to, the 5′-exonuclease assay TaqMan® labeling probes described herein (see also U.S. Pat. No. 5,538,848), various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons' (see, e.g., U.S. Pat. Nos. 6,355,421 and 6,593,091), linear PNA beacons (see, e.g., Kubista et al., 2001, SPIE 4264:53-58), non-FRET labeling probes (see, e.g., U.S. Pat. No. 6,150,097), Sunrise®/Amplifluor® labeling probes (U.S. Pat. No. 6,548,250), stem-loop and duplex Scorpion™ labeling probes (Solinas et al., 2001, Nucleic Acids Research 29:E96 and U.S. Pat. No. 6,589,743), bulge loop labeling probes (U.S. Pat. No. 6,590,091), pseudo knot labeling probes (U.S. Pat. No. 6,589,250), cyclicons (U.S. Pat. No. 6,383,752), MGB Eclipse™ probe (Epoch Biosciences), hairpin labeling probes (U.S. Pat. No. 6,596,490), peptide nucleic acid (PNA) light-up labeling probes, self-assembled nanoparticle labeling probes, and ferrocene-modified labeling probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Labeling probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Labeling probes can also comprise black hole quenchers (Biosearch), Iowa Black (IDT), QSY quencher (Molecular Labeling probes), and Dabsyl and Dabcel sulfonate/carboxylate Quenchers (Epoch). Labeling probes can also comprise two labeling probes, wherein for example a fluorophore is on one probe, and a quencher on the other, wherein hybridization of the two labeling probes together on a target quenches the signal, or wherein hybridization on target alters the signal signature via a change in fluorescence. Labeling probes can also comprise sulfonate derivatives of fluorescenin dyes with a sulfonic acid group instead of the carboxylate group, phosphoramidite forms of fluorescein, phosphoramidite forms of CY 5 (available for example from Amersham).

The term “ionizing radiation” refers to radiation that has sufficient energy to eject one or more orbital electrons from an atom or molecule (e.g. a particles, β particles, γ rays, x-rays, neutrons, protons and other particles having sufficient energy to produce ion pairs in matter).

The term “at risk of exposure” as used herein refers to a subject who is believed to be exposed or has been subjected to conditions that could lead to exposure, of ionizing radiation.

Extracellular miRNAs freely circulate in a wide range of bodily fluids. Accordingly, in some embodiments, the biological sample used for determining the level of one or more miRNA biomarkers is a bodily fluid, such as blood, fractions thereof, serum, plasma, urine, saliva, tears, sweat, semen, vaginal secretions, lymph, bronchial secretions, or CSF. In some embodiments, the sample is a sample that is obtained non-invasively. In some embodiments, the sample is obtained from a bodily fluid other than CSF. In some embodiments, the biological sample used for determining the level of one or more miRNA biomarkers may contain cells. In other embodiments, the biological sample may be free or substantially free of cells (e.g., a serum or plasma sample). The sample may likewise be free or substantially free of microvesicles. For example, a sample that is free or substantially free of microvesicles is one in which the microvesicle content of the sample is sufficiently low to avoid interfering with the ability to accurately determine the level of non-vesicular miRNAs in the sample.

A number of embodiments of the invention have been described. Nevertheless, it will be understood that various modifications may be made without departing from the spirit and scope of the invention. Accordingly, other embodiments are within the scope of the following claims.

In certain embodiments, the present invention is directed to a panel of time agnostic lethal radiation markers that include microRNAs (miRNAs), which are sequentially conserved between human and minipig. Embodiments provide a unique panel of biomarkers, which provide a reliable resource to assess the stress history, since miRNAs can control both transcription and translational mechanisms, and regulate epigenomic profiles.

Furthermore, also disclosed are methods to curate biomarkers that are not only effective in finding markers with high clinical potential, but also embody a novel in silico method. This protocol showed an aptitude to identify those miRNAs markers, which not only share high sequence homology between human and minipig, but also present a cross-species conserved functional response to lethal radiation.

### Biomarkers

Also disclosed are methods for measuring exposure of a human subject to ionizing radiation involving the detection of one or more biomarkers. Following list of markers possess one common feature, namely their sequences are conserved between human and minipig.

A. Time independent marker: The method comprises the assaying of biosample (e.g. serum, plasma, or other cell free biosample obtained from a subject) to find following miRNAs, which are significantly down regulated from the normal non-irradiated human serum/plasma samples starting immediately (e.g. approximately 0-4 months of human life) after the radiation up till nearly 12 months of human life post radiation: miR-432-5p, miR-221-5p, miR-92b-5p, miR-331-3p, miR-424-5p, miR-376a-3p and miR-374b-5p.

B. Early markers:

B. i. The method comprises the assaying of biosample to find following miRNAs, which are significantly upregulated from the normal non-irradiatedbiosample samples immediately (e.g. approximately 0-4 months of human life) post-irradiation: miR-218-5p, miR-96-5p, miR-671-5p, miR-424-3p, miR-490-3p, miR-296-5p, miR-769-3p, miR-27b-5p, miR-190b, miR-374b-3p, miR-193a-3p, miR-202-3p, miR-885-3p and miR-217.

B. ii. The method comprises the assaying of biosamples to find following miRNAs, which are significantly down-regulated from the normal non-irradiated serum/plasma samples immediately (e.g. 0-4 months of human life) post-radiation: miR-532-3p, miR-885-5p, miR-545-5p, miR-1296-5p, miR-133b, miR-206, miR-432-5p, miR-221-5p, miR-92b-5p, miR-331-3p, miR-424-5p, miR-30c-1-3p, miR-676-3p, miR-542-3p, miR-542-5p, miR-184, miR-193a-5p, miR-376a-3p, miR-374b-5p, miR-139-5p, miR-545-3p, miR-133a-3p, miR-133a-5p and miR-1306-3p.

C. Intermediate marker:

C. i. The method comprises the assaying of biosamples to find following miRNAs, which are significantly upregulated from the normal non-irradiated biosamples between 5-9 months of human life post radiation: miR-218-5p, miR-190b, miR-339-3p, miR-429 and miR-676-3p.

C. ii. The method comprises the assaying of biosamples to find following miRNAs, which are significantly down-regulated from the normal non-irradiated serum/plasma samples between 5-9 months of human life post radiation: let-7d-5p, miR-1306-5p, miR-139-3p, miR-140-5p, miR-142-3p, miR-142-5p, miR-145-3p, miR-148a-5p, miR-148a-5p, miR-148b-5p, miR-155-5p, miR-17-3p, miR-17-5p, miR-181d-5p, miR-199a-5p, miR-199b-5p, miR-221-3p, miR-22-3p, miR-22-5p, miR-24-1-5p, miR-24-3p, miR-27b-3p, miR-28-3p, miR-28-5p, miR-296-3p, miR-30a-3p, miR-30b-5p, miR-30c-5p, miR-30e-3p, miR-326, miR-331-5p, miR-345-3p, miR-361-3p, miR-361-5p, miR-374a-3p, miR-374a-5p, miR-425-3p, miR-425-5p, mir-450b-5p, miR-455-3p, miR-455-5p, miR-532-5p, miR-671-3p, miR-6782-3p, miR-769-5p, miR-1296-5p, miR-133b, miR-542-5p, miR-184, miR-193a-5p, miR-545-3p, miR-133a-3p, miR-432-5p, miR-221-5p, miR-92b-5p, miR-331-3p, miR-424-5p, miR-30c-1-3p, miR-542-3p, miR-376a-3p, miR-374b-5p, miR-139-5p, miR-545-3p, miR-133a-3p, miR-133a-5p and miR-1306-3p.

D. Late markers:

D. i. The method comprises the assaying of biosamples to find following miRNAs, which are significantly upregulated from the normal non-irradiatedbiosamples approximately 12 months of human life post-irradiation: miR-218-5p, miR-296-5p, miR-126-5p, miR-424-3p, miR-143-3p, miR-345-3p, miR-708-3p and miR-708-5p.

D. ii. The method comprises the assaying of biosamples to find following miRNAs, which are significantly down-regulated from the normal non-irradiated serum/plasma samples approximately 12 months of human life post-irradiation: miR-181d-5p, miR-24-1-5p, miR-331-5p, miR-432-5p, miR-221-5p, miR-92b-5p, miR-331-3p, miR-424-5p, miR-376a-3p, miR-374b-5p and miR-1306-3p.

Detection of Biomarkers

The level of one or more miRNA biomarkers described herein in a biological sample may be determined by any suitable method. Any reliable method for measuring the level or amount of miRNA in a sample may be used. Generally, miRNA can be detected and quantified from a sample (including fractions thereof), such as samples of isolated RNA by various methods known for mRNA detection, including, for example, amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g., hybridization arrays (e.g., microarrays), NanoString™ analysis, Northern Blot analysis, branched DNA (bDNA) signal amplification, and in situ hybridization), and sequencing-based methods (e.g. next-generation sequencing methods, for example, using the Illumina or IonTorrent platforms). Other exemplary techniques include ribonuclease protection assay (RPA) and mass spectroscopy.

In some embodiments, RNA is converted to complementary DNA (cDNA) prior to analysis. cDNA can be generated by reverse transcription of isolated RNA or miRNA using conventional techniques. Reverse transcription kits are known and commercially available. Examples of suitable kits include, but are not limited to the mirVana TaqMan® miRNA transcription kit (Ambion, Austin, Tex.), and the TaqMan® miRNA transcription kit (Applied Biosystems, Foster City, Calif.). Universal primers, or specific primers, including miRNA-specific stem-loop primers, are known and commercially available, for example, from Applied Biosystems. In some embodiments, miRNA is amplified prior to measurement. In other embodiments, the level of miRNA is measured during the amplification process. In still other embodiments, the level of miRNA is not amplified prior to measurement. Some exemplary methods suitable for determining the level of miRNA in a sample are described in greater detail below. These methods are provided by way of illustration only, and it will be apparent to a skilled person that other suitable methods may likewise be used.

Many amplification-based methods exist for detecting the level of miRNA nucleic acid sequences, including, but not limited to, PCR, RT-PCR, qPCR, and rolling circle amplification. Other amplification-based techniques include, for example, ligase chain reaction, multiplex ligatable probe amplification, in vitro transcription (IVT), strand displacement amplification, transcription-mediated amplification, RNA (Eberwine) amplification, and other methods that are known to persons skilled in the art.

A typical PCR reaction includes multiple steps, or cycles, that selectively amplify target nucleic acid species: a denaturing step, in which a target nucleic acid is denatured; an annealing step, in which a set of PCR primers (i.e., forward and reverse primers) anneal to complementary DNA strands, and an elongation step, in which a thermostable DNA polymerase elongates the primers. By repeating these steps multiple times, a DNA fragment is amplified to produce an amplicon, corresponding to the target sequence. Typical PCR reactions include 20 or more cycles of denaturation, annealing, and elongation. In many cases, the annealing and elongation steps can be performed concurrently, in which case the cycle contains only two steps. A reverse transcription reaction (which produces a cDNA sequence having complementarity to a miRNA) may be performed prior to PCR amplification. Reverse transcription reactions include the use of, e.g., a RNA-based DNA polymerase (reverse transcriptase) and a primer. Kits for quantitative real time PCR of miRNA are known, and are commercially available. Examples of suitable kits include, but are not limited to, the TaqMan® miRNA Assay (Applied Biosystems) and the mirVana™ qRT-PCR miRNA detection kit (Ambion). The miRNA can be ligated to a single stranded oligonucleotide containing universal primer sequences, a polyadenylated sequence, or adaptor sequence prior to reverse transcriptase and amplified using a primer complementary to the universal primer sequence, poly(T) primer, or primer comprising a sequence that is complementary to the adaptor sequence.

In some instances, custom qRT-PCR assays can be developed for determination of miRNA levels. Custom qRT-PCR assays to measure miRNAs in a biological sample, e.g., a body fluid, can be developed using, for example, methods that involve an extended reverse transcription primer and locked nucleic acid modified PCR. Custom miRNA assays can be tested by running the assay on a dilution series of chemically synthesized miRNA corresponding to the target sequence. This permits determination of the limit of detection and linear range of quantitation of each assay. Furthermore, when used as a standard curve, these data permit an estimate of the absolute abundance of miRNAs measured in biological samples.

Amplification curves may optionally be checked to verify that Ct values are assessed in the linear range of each amplification plot. Typically, the linear range spans several orders of magnitude. For each candidate miRNA assayed, a chemically synthesized version of the miRNA can be obtained and analyzed in a dilution series to determine the limit of sensitivity of the assay, and the linear range of quantitation. Relative expression levels may be determined, for example, according to the 2(−ΔΔC(T)) Method.

In some embodiments, two or more miRNAs are amplified in a single reaction volume. For example, multiplex q-PCR, such as qRT-PCR, enables simultaneous amplification and quantification of at least two miRNAs of interest in one reaction volume by using more than one pair of primers and/or more than one probe. The primer pairs comprise at least one amplification primer that specifically binds each miRNA, and the probes are labeled such that they are distinguishable from one another, thus allowing simultaneous quantification of multiple miRNAs.

Rolling circle amplification is a DNA-polymerase driven reaction that can replicate circularized oligonucleotide probes with either linear or geometric kinetics under isothermal conditions. In the presence of two primers, one hybridizing to the (+) strand of DNA, and the other hybridizing to the (−) strand, a complex pattern of strand displacement results in the generation of over 10⁹ copies of each DNA molecule in 90 minutes or less. Tandemly linked copies of a closed circle DNA molecule may be formed by using a single primer. The process can also be performed using a matrix-associated DNA. The template used for rolling circle amplification may be reverse transcribed. This method can be used as a highly sensitive indicator of miRNA sequence and expression level at very low miRNA concentrations.

miRNA may also be detected using hybridization-based methods, including but not limited to hybridization arrays (e.g., microarrays), NanoString™ analysis, Northern Blot analysis, branched DNA (bDNA) signal amplification, and in situ hybridization.

Microarrays can be used to measure the expression levels of large numbers of miRNAs simultaneously. Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays. Also useful are microfluidic TaqMan Low-Density Arrays, which are based on an array of microfluidic qRT-PCR reactions, as well as related microfluidic qRT-PCR based methods.

In one example of microarray detection, various oligonucleotides (e.g., 200+5′-amino-modified-C6 oligos) corresponding to human sense miRNA sequences are spotted on three-dimensional CodeLink slides (GE Health/Amersham Biosciences) at a final concentration of about 20 μM and processed according to manufacturer's recommendations. First strand cDNA synthesized from 20 μg TRIzol-purified total RNA is labeled with biotinylated ddUTP using the Enzo BioArray end labeling kit (Enzo Life Sciences Inc.). Hybridization, staining, and washing can be performed according to a modified Affymetrix Antisense genome array protocol.

Axon B-4000 scanner and Gene-Pix Pro 4.0 software or other suitable software can be used to scan images. Non-positive spots after background subtraction, and outliers detected by the ESD procedure, are removed. The resulting signal intensity values may be normalized to per-chip median values and then used to obtain geometric means and standard errors for each miRNA. Each miRNA signal can be transformed to log base 2, and a one-sample t test can be conducted. Independent hybridizations for each sample can be performed on chips with each miRNA spotted multiple times to increase the robustness of the data.

Microarrays can be used for the expression profiling of miRNAs in diseases. For example, RNA can be extracted from a sample and, optionally, the miRNAs are size-selected from total RNA. Oligonucleotide linkers can be attached to the 5′ and 3′ ends of the miRNAs and the resulting ligation products are used as templates for an RT-PCR reaction. The sense strand PCR primer can have a fluorophore attached to its 5′ end, thereby labeling the sense strand of the PCR product. The PCR product is denatured and then hybridized to the microarray. A PCR product, referred to as the target nucleic acid that is complementary to the corresponding miRNA capture probe sequence on the array will hybridize, via base pairing, to the spot at which the, capture probes are affixed. The spot will then fluoresce when excited using a microarray laser scanner.

The fluorescence intensity of each spot is then evaluated in terms of the number of copies of a particular miRNA, using a number of positive and negative controls and array data normalization methods, which will result in assessment of the level of expression of a particular miRNA.

Total RNA containing the miRNA extracted from a body fluid sample can also be used directly without size-selection of the miRNAs. For example, the RNA can be 3′ end labeled using T4 RNA ligase and a fluorophore-labeled short RNA linker. Fluorophore-labeled miRNAs complementary to the corresponding miRNA capture probe sequences on the array hybridize, via base pairing, to the spot at which the capture probes are affixed. The fluorescence intensity of each spot is then evaluated in terms of the number of copies of a particular miRNA, using a number of positive and negative controls and array data normalization methods, which will result in assessment of the level of expression of a particular miRNA.

Several types of microarrays can be employed including, but not limited to, spotted oligonucleotide microarrays, pre-fabricated oligonucleotide microarrays or spotted long oligonucleotide arrays.

miRNAs can also be detected without amplification using the nCounter® Analysis System (NanoString™ Technologies, Seattle, Wash.). This technology employs two nucleic acid-based probes that hybridize in solution (e.g., a reporter probe and a capture probe). After hybridization, excess probes are removed, and probe/target complexes are analyzed in accordance with the manufacturer's protocol. nCounter® miRNA assay kits are available from NanoString™ Technologies, which are capable of distinguishing between highly similar miRNAs with great specificity. miRNAs can also be detected using branched DNA (bDNA) signal amplification (see, for example, Urdea, Nature Biotechnology (1994), 12:926-928). miRNA assays based on bDNA signal amplification are commercially available. One such assay is the QuantiGene® 2.0 miRNA Assay (Affymetrix, Santa Clara, Calif.).

Northern Blot and in situ hybridization may also be used to detect miRNAs. Suitable methods for performing Northern Blot and in situ hybridization are known in the art.

Advanced sequencing methods can likewise be used as available. For example, miRNAs can be detected using Illumina® Next Generation Sequencing (e.g., Sequencing-By-Synthesis or TruSeq methods, using, for example, the HiSeq, HiScan, GenomeAnalyzer, or MiSeq systems (Illumina, Inc., San Diego, Calif.)). miRNAs can also be detected using Ion Torrent Sequencing (Ion Torrent Systems, Inc., Gulliford, Conn.), or other suitable methods of semiconductor sequencing.

Mass spectroscopy can also be used to quantify miRNA using RNase mapping. Isolated RNAs can be enzymatically digested with RNA endonucleases (RNases) having high specificity (e.g., RNase Tl, which cleaves at the 3′-side of all unmodified guanosine residues) prior to their analysis by MS or tandem MS (MS/MS) approaches. The first approach developed utilized the on-line chromatographic separation of endonuclease digests by reversed phase HPLC coupled directly to ESTMS. The presence of posttranscriptional modifications can be revealed by mass shifts from those expected based upon the RNA sequence. Ions of anomalous mass/charge values can then be isolated for tandem MS sequencing to locate the sequence placement of the posttranscriptionally modified nucleoside.

Matrix-assisted laser desorption/ionization mass spectrometry (MALDI-MS) has also been used as an analytical approach for obtaining information about post-transcriptionally modified nucleosides. MALDI-based approaches can be differentiated from EST-based approaches by the separation step. In MALDI-MS, the mass spectrometer is used to separate the miRNA.

To analyze a limited quantity of intact miRNAs, a system of capillary LC coupled with nanoESI-MS can be employed, by using a linear ion trap-orbitrap hybrid mass spectrometer (LTQ Orbitrap XL, Thermo Fisher Scientific) or a tandem-quadrupole time-of-flight mass spectrometer (QSTAR® XL, Applied Biosystems) equipped with a custom-made nanospray ion source, a Nanovolume Valve (Valco Instruments), and a splitless nano HPLC system (DiNa, KYA Technologies). Analyte/TEAA is loaded onto a nano-LC trap column, desalted, and then concentrated. Intact miRNAs are eluted from the trap column and directly injected into a CI 8 capillary column, and chromatographed by RP-HPLC using a gradient of solvents of increasing polarity. The chromatographic eluent is sprayed from a sprayer tip attached to the capillary column, using an ionization voltage that allows ions to be scanned in the negative polarity mode.

Additional methods for miRNA detection and measurement include, for example, strand invasion assay (Third Wave Technologies, Inc.), surface plasmon resonance (SPR), cDNA, MTDNA (metallic DNA; Advance Technologies, Saskatoon, SK), and single-molecule methods such as the one developed by US Genomics. Multiple miRNAs can be detected in a microarray format using a novel approach that combines a surface enzyme reaction with nanoparticle-amplified SPR imaging (SPRI). The surface reaction of poly(A) polymerase creates poly(A) tails on miRNAs hybridized onto locked nucleic acid (LNA) microarrays. DNA-modified nanoparticles are then adsorbed onto the poly(A) tails and detected with SPRI. This ultrasensitive nanoparticle-amplified SPRI methodology can be used for miRNA profiling at attamole levels.

In certain embodiments, labels, dyes, or labeled probes and/or primers are used to detect amplified or unamplified miRNAs. The skilled artisan will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where miRNA amplification is preferred.

A probe or primer may include standard (A, T or U, G and C) bases, or modified bases. Modified bases include, but are not limited to, the AEGIS bases (from Eragen Biosciences). In certain aspects, bases are joined by a natural phosphodiester bond or a different chemical linkage. Different chemical linkages include, but are not limited to, a peptide bond or a Locked Nucleic Acid (LNA) linkage.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay {e.g., TaqMan™) probes, stem-loop molecular beacons, stemless or linear beacons, peptide nucleic acid (PNA) Molecular Beacons, linear PNA beacons, non-FRET probes, Sunrise™/AmplifluorB™ probes, stem-loop and duplex Scorpion™ probes, bulge loop probes, pseudo knot probes, cyclicons, MGB Eclipse™ probe (Epoch Biosciences), hairpin probes, PNA light-up probes, anti-primer quench probes, self-assembled nanoparticle probes, and ferrocene-modified probes.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more distinguishable labels. In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g. biotin-avidin). In still other aspects, use of labels can be accomplished using any one of a large number of known techniques employing known labels, linkages, linking groups, reagents, reaction conditions, and analysis and purification methods.

miRNAs can be detected by direct or indirect methods. In a direct detection method, one or more miRNAs are detected by a detectable label that is linked to a nucleic acid molecule. In such methods, the miRNAs may be labeled prior to binding to the probe. Therefore, binding is detected by screening for the labeled miRNA that is bound to the probe. The probe is optionally linked to a bead in the reaction volume.

In certain embodiments, nucleic acids are detected by direct binding with a labeled probe, and the probe is subsequently detected. In one embodiment of the invention, the nucleic acids, such as amplified miRNAs, are detected using FlexMAP Microspheres (Luminex) conjugated with probes to capture the desired nucleic acids. Some methods may involve detection with polynucleotide probes modified with fluorescent labels or branched DNA (bDNA) detection, for example.

In other embodiments, nucleic acids are detected by indirect detection methods. For example, a biotinylated probe may be combined with a streptavidin-conjugated dye to detect the bound nucleic acid. The streptavidin molecule binds a biotin label on amplified miRNA, and the bound miRNA is detected by detecting the dye molecule attached to the streptavidin molecule. In one embodiment, the streptavidin-conjugated dye molecule comprises Phycolink® Streptavidin R-Phycoerythrin (PROzyme). Other conjugated dye molecules are known to persons skilled in the art.

Labels include, but are not limited to: light-emitting, light-scattering, and light-absorbing compounds which generate or quench a detectable fluorescent, chemiluminescent, or bioluminescent signal. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In certain embodiments, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups.

In other embodiments, methods relying on hybridization and/or ligation to quantify miRNAs may be used, including oligonucleotide ligation (OLA) methods and methods that allow a distinguishable probe that hybridizes to the target nucleic acid sequence to be separated from an unbound probe. As an example, HARP-like probes may be used to measure the quantity of miRNAs. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified.

A probe ligation reaction may also be used to quantify miRNAs. In a Multiplex Ligation-dependent Probe Amplification (MLPA) technique, pairs of probes which hybridize immediately adjacent to each other on the target nucleic acid are ligated to each other driven by the presence of the target nucleic acid. In some aspects, MLPA probes have flanking PCR primer binding sites. MLPA probes are specifically amplified when ligated, thus allowing for detection and quantification of miRNA biomarkers.

### Radiation Injury Treatment

The term “radiation injury therapy” refers to the medical management of a patient with the intent to cure, ameliorate, stabilize, or prevent a radiation injury or symptoms thereof.

ARS, also known as radiation poisoning, radiation sickness, or radiation toxicity, is a constellation of health effects which present within 24 hours of exposure to high amounts of ionizing radiation. The radiation causes cellular degradation due to damage to DNA and other key molecular structures within the cells in various tissues; this destruction, particularly as it affects ability of cells to divide normally, in turn causes the symptoms. The symptoms can begin within one or two hours and may last for several months. The terms refer to acute medical problems rather than ones that develop after a prolonged period. The onset and type of symptoms depends on the radiation exposure. Relatively smaller doses result in gastrointestinal effects such as nausea and vomiting and symptoms related to falling blood counts such as infection and bleeding. Relatively larger doses can result in neurological effects and rapid death.

Similar symptoms may appear months to years after exposure as chronic radiation syndrome when the dose rate is too low to cause the acute form or as delayed or late effects of the acute exposure. Radiation exposure can also increase the probability of developing some other diseases, mainly different types of cancers. These diseases are sometimes referred to as radiation sickness, but they are never included in the term acute radiation syndrome.

Classically acute radiation syndrome can affect the hematopoietic, gastrointestinal, pulmonary, and neurological/vascular systems. These symptoms may or may not be preceded by a prodrome. The speed of onset of symptoms is related to radiation exposure, with greater doses resulting in a shorter delay in symptom onset. These presentations presume whole-body exposure and many of them are markers which are not valid if the entire body has not been exposed. Each syndrome requires that the tissue showing the syndrome itself be exposed. The hematopoietic syndrome requires exposure of the areas of bone marrow actively forming blood elements (i.e., the pelvis and sternum in adults). The neurovascular symptoms require exposure of the brain. The gastrointestinal syndrome is not seen if the stomach and intestines are not exposed to radiation.

The hematopoietic syndrome is marked by a drop in the number of blood cells, called aplastic anemia. This may result in infections due to low white blood cells, bleeding due to low platelets, and anemia due to low red blood cells. These changes can be detected by blood tests after receiving a whole-body acute dose as low as 0.25 Gy, though they might never be felt by the patient if the dose is below 1 Gy. Conventional trauma and burns resulting from a bomb blast are complicated by the poor wound healing caused by hematopoietic syndrome, increasing mortality.

The gastrointestinal syndrome often follows absorbed doses of 6-30 Gy. Nausea, vomiting, loss of appetite, and abdominal pain are usually seen within two hours. Vomiting in this time-frame is a marker for whole body exposures that are in the fatal range above 4 Gy.

The neurovascular syndrome typically occurs at absorbed doses greater than 30 Gy, though it may occur at 10 Gy. It presents with neurological symptoms such as dizziness, headache, or decreased level of consciousness, occurring within minutes to a few hours, and with an absence of vomiting. It is invariably fatal.

Radiation induced lung injury can lead to pneumonitis (interstitial pulmonary inflammation) in 1-6 months. This often leads to fibrosis (scaring, collagen deposition) in 6 months to several years. Penumonitis and fibrosis causes respiratory distress and even death. Thoracic irradiation can also lease to lung cancer, breast cancer, lymphoma, etc.

The prodrome (early symptoms) of ARS typically includes nausea and vomiting, headaches, fatigue, fever, and short period of skin reddening. These symptoms may occur at radiation doses as low as 35 rads. These symptoms are common to many illnesses and may not, by themselves, indicate acute radiation sickness.

In the event of a large scale radiation accident or nuclear attack, a large number of individuals will need to be triaged to determine their dose of radiation exposure. The disclosed dosimeters and methods can be used to identify individuals who need treatment and those who do not. NIH guidelines indicate that individuals that receive 2 Gy and above need treatment. The DoD indicates that when combined with wound and burn, the critical dose for triage (to treat or not to treat) is 1.5 Gy.

Treatment is generally supportive with the use of antibiotics, blood products, colony stimulating factors, and stem cell transplant as clinically indicated. Symptomatic measures may also be employed. However, it is important to identify the organ(s) affected by the radiation and the dose they received in order to select the appropriate therapy for the subject.

For example, if it is determined that the hematopoietic system has been affected, then the subject can be treated with hematopoietic stem cell transplant, blood transfusion, or administration of growth factors, such as GM-CSF (Neupogen). In some cases, this treatment should occur within few days of whole body exposure of a significant dose (e.g., 2 Gy and above) or a partial body exposure to a significant dose (e.g., 4 Gy and above) with significant bone marrow coverage.

If it is determined that the GI track has been affected, the subject can be treated with intestinal stem cell therapy.

If it is determined that the lung has been affected, the subject can be treated with antioxidants and/or superoxide dismutase mimetics (e.g. AEOL 101050, a metalloporphyrin antioxidant developed by Aeolus). Corticosteroids (e.g. Dexamethasone) can be given to patients to subside penumonitis. The disclosed methods can identify individuals at risk of pneumonitis and thereby allow early administration of corticosteroids before the expression of the issue. Also the panel will help in testing the efficacy of mitigators.

The treatment of established or suspected infection following exposure to radiation (characterized by neutropenia and fever) is similar to the one used for other febrile neutropenic patients. However, important differences between the two conditions exist. Individuals that develop neutropenia after exposure to radiation are also susceptible to irradiation damage in other tissues, such as the gastrointestinal tract, lungs and central nervous system. These patients may require therapeutic interventions not needed in other types of neutropenic patients. The response of irradiated animals to antimicrobial therapy can be unpredictable, as was evident in experimental studies where metronidazole and pefloxacin therapies were detrimental. Antimicrobials that reduce the number of the strict anaerobic component of the gut flora (i.e., metronidazole) generally should not be given because they may enhance systemic infection by aerobic or facultative bacteria, thus facilitating mortality after irradiation.

An empirical regimen of antimicrobials can be chosen based on the pattern of bacterial susceptibility and nosocomial infections in the affected area and medical center and the degree of neutropenia. Broad-spectrum empirical therapy with high doses of one or more antibiotics can be initiated at the onset of fever. These antimicrobials can be directed at the eradication of Gram-negative aerobic bacilli that account for more than three quarters of the isolates causing sepsis. Because aerobic and facultative Gram-positive bacteria (mostly alpha-hemolytic streptococci) cause sepsis in about a quarter of the victims, coverage for these organisms may also be needed.

A standardized management plane of febrile, neutropenic patients must be devised in each institution or agency. Empirical regimens must contain antibiotics broadly active against Gram-negative aerobic bacteria (quinolones: i.e., ciprofloxacin, levofloxacin, a third- or fourth-generation cephalosporin with pseudomonal coverage: e.g., cefepime, ceftazidime, or an aminoglycoside: i.e. gentamicin, amikacin).

The anti-clotting compounds thrombomodulin (Solulin/Recomodulin) and activated protein C (Xigris) have also been shown to increase bone marrow cells needed for the production of white blood cells, and improve the survival rates of mice receiving lethal radiation doses by 40-80%.

Additionally, thrombopoietic activities of the glycosylflavanoids Orientin and Vicenin can be used to enhance the reconstitution of circulating platelets.

In some cases, hydration and palliative care is selected for subjects determined to have been exposed to lethal, irremediable doses of radiation.

### EXAMPLES

### Example 1: Materials and Methods

Animal Husbandry

Three male Gottingen minipigs (Sus scrofa domestics) were acquired from Marshall BioResources (North Rose, NY) between ages 3-5 months and weighing 8.5-10.5 kg. Pigs were individually housed (with social contact maintained through slatted cages) in the AFRRI Veterinary Sciences Department (VSD) vivarium on arrival and were quarantined and acclimatized for two weeks. Water was provided ad libitum and twice daily meal portions (Mini-Swine Diet 8753, Envigo Teklad Diets, Madison, WI) were determined by supplier instruction according to animal weight.

Irradiation and Dosimetry

Following the quarantine period, GMPs were exposed to 2.2 Gy⁶⁰ Cobalt (⁶⁰Co) total body irradiation (dose rate ˜0.6 Gy/minute; unilateral sequential raising of the two ⁶⁰Co sources) in the AFRRI Cobalt Radiation Facility. Dosimetry was confirmed as described previously³¹. Anesthesia for irradiation was as follows: 5-2% isoflurane supplied by mask (5% for induction; 2% for maintenance) followed by IM injection of TelazolVR (100 mg/mL, 2 mg/kg) and Xylazine (50 mg/mL, 1 mg/kg). Post-irradiation, pigs were monitored closely until fully ambulatory.

Blood Collection

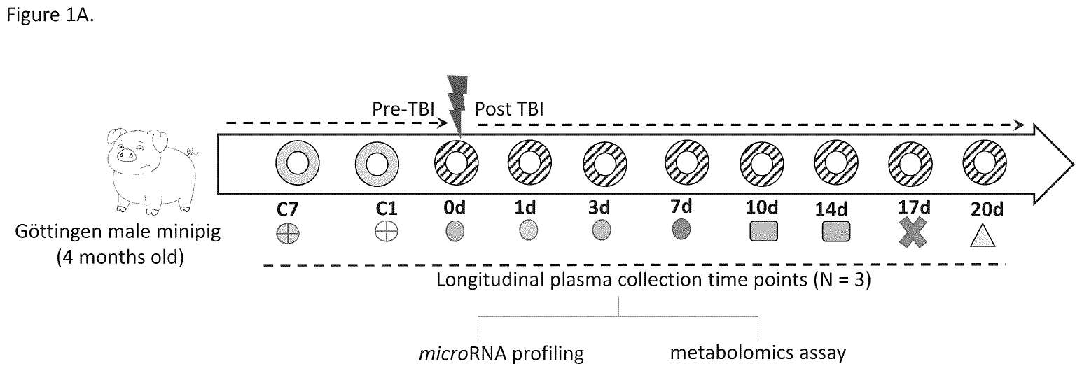

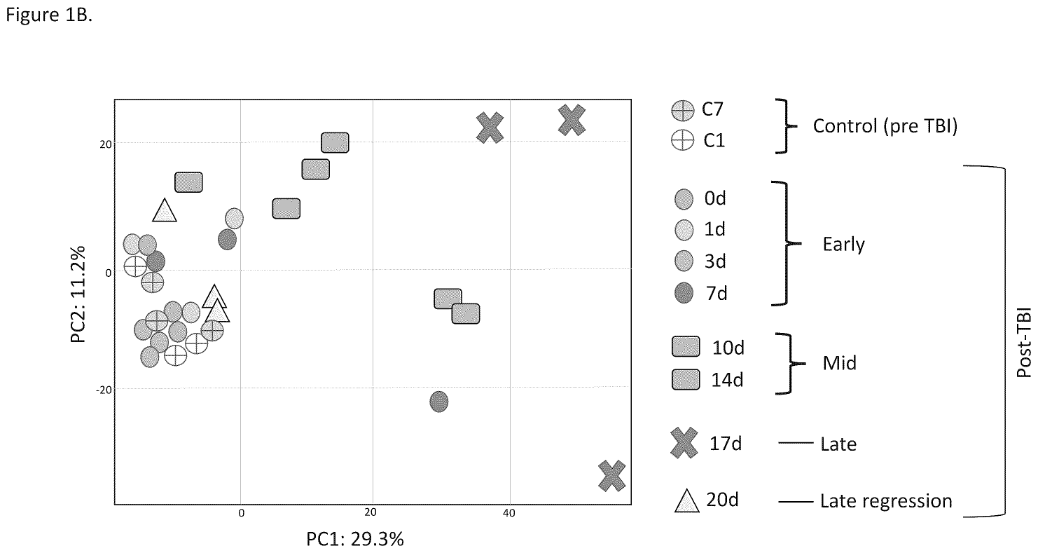

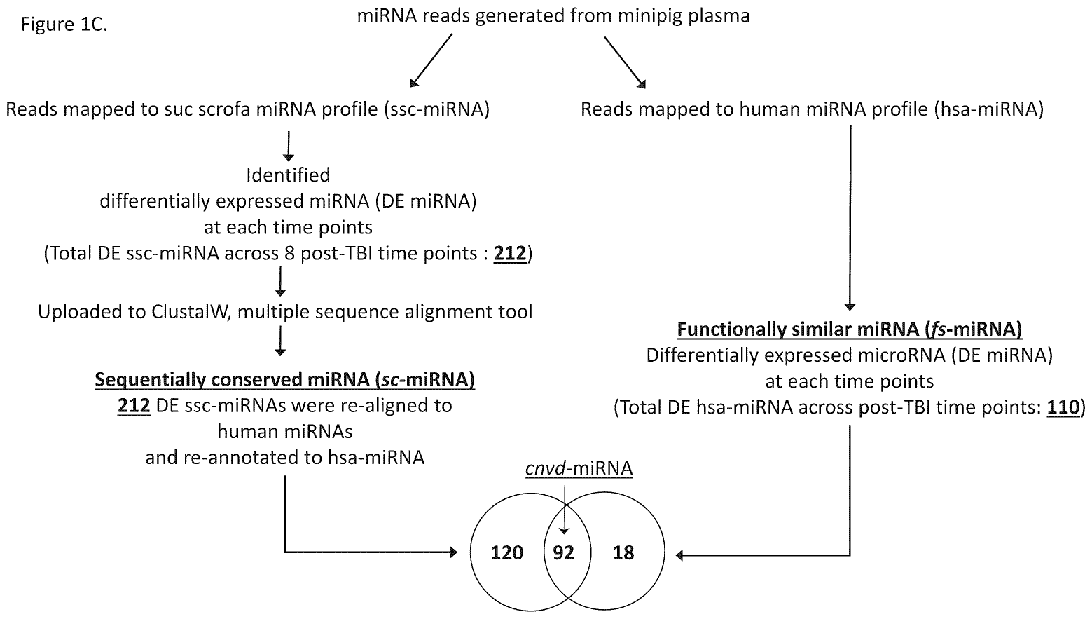

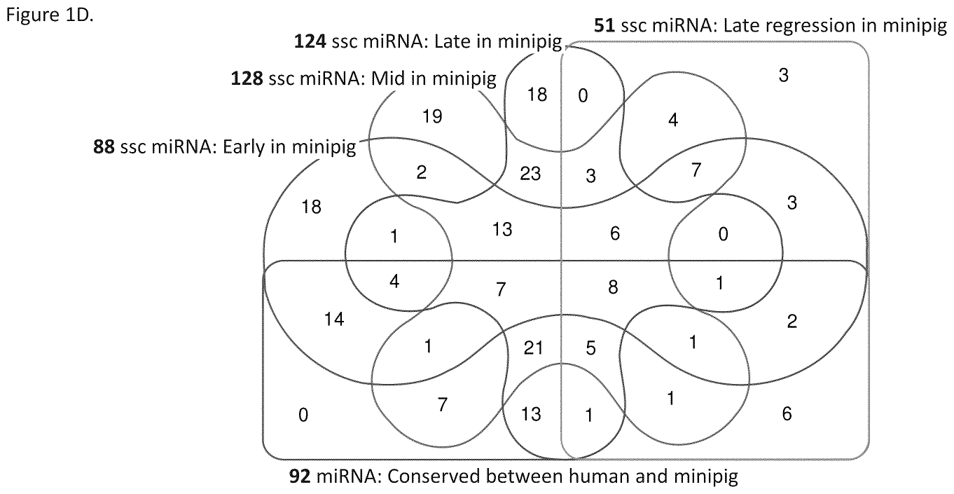

Animals were anesthetized with 5-2% isoflurane (as described above) and blood was collected from peripheral veins into anticoagulated tubes on the day of irradiation (day 0) and days 1, 3, 7, 10, 14, 17, and 20 following exposure (FIG. 1A). Baseline values were established in blood collected 7 and 1 days prior to irradiation. The time points were categorized into four phases, Early (days 0, 1, 3 and 7), Mid (days 10 and 14), Late (day 17), and LR (day 20) of H-ARS. Complete blood counts were determined in whole blood; plasma was separated by centrifugation and stored at −80° C. until use.

Treatment, Monitoring, and Supportive Care

Days 1 and 8 post-TBI, GMPs received 5% dextrose subcutaneously (animals comprised control group of a countermeasure testing study³⁰; injection volume was equivalent to volume of drug injected). Indicators of animal health (temperature, activity, posture, stool, vomit, respiratory activity and rate, and anorexia) were monitored twice daily. At time of blood collections, heart rate, lung sounds, perfusion, hydration, and body weight were assessed. Supportive care (prophylactic ciprofloxacin and amoxicillin/clavulanate (p.o.)) was administered when absolute neutrophil counts were <500/μL as described previously²⁷.

Euthanasia

During daily health checks, GMPs were also monitored for pre-determined criteria that necessitated unscheduled euthanasia: detection of one absolute criteria (non-responsiveness, dyspnea, or hypothermia) or four or more non-absolute criteria (hyperthermia, anorexia, anemia, vomiting/diarrhea, lethargy, vestibular signs, prolonged hemorrhage (bruising, petechiae, frank bleeding)). Anesthetized animals (5-2% isoflurane and Xylazine and Telazol IM injection (as described above)) were euthanized with an IV injection of Euthasol® (sodium pentobarbital; 1 mL/4.5 kg).

miRNA Sequencing Analysis

Five microliter (μL) of GMP plasma sample ligated with adapters to construct the sequencing libraries using the TruSeq small RNA Sample Preparation kit and a multiplexed pool consisting of equimolar amounts of small RNA-derived libraries was size selected. Illumina NextSeq platform was used to generate 5 million reads for miRNA profiling⁵³. Preprocessing of raw base calls and sample de-multiplexing were performed using the standard open-source tool bcl2fastq2 v2.20 (Illumina Inc., San Diego, CA, USA). Reads were quality filtered, adaptor trimmed in silico using Cutadapt software v1.16, and mapped against Sus scrofa v5 (ssc5) reference genome assembly using Bowtie v2.2.3.0 aligner. Next, these mapped reads were re-mapped to the established Sus scrofa miRNAs (ssc-miRNA) that was archived in miRBase. Using miRDeep2 core algorithm, we conducted ssc-miRNA detection and de novo ssc-miRNA prediction from ss5 reference genome assembly of minipig. The known ssc-miRNA quantification was determined using miRDeep2 quantification function. Count data was further analyzed using edgeR v3.6 downloaded from R Bioconductor package. ssc-miRNA reads less than 5 read counts (cpm>0.5) in 10% of sample population were filtered out and the remaining counts were normalized by TMM normalization method after library size factor calculations. Sample library size distribution and per sample ssc-miRNA read counts matrix were generated to see overall quality of the data and Principal component analysis (PCA) of the ssc-miRNAs was conducted to uncover longitudinal impact of TBI (FIG. 1B).

Differential expression (DE) analysis was performed using edgeR, where data was fit under negative binomial generalized log-linear model to the read counts for each miRNAs. The baseline was the pool of three pre-TBI time points, and the gene pair-wise analysis identified DE ssc-miRNAs at each time points, at the cut-off moderate t-test p<0.05.

Subsequent analysis was focused on finding DE ssc-miRNAs, which would be sequentially conserved and functionally similar between human and minipig exposed to TBI (FIG. 1C). Briefly, we pooled all of these DE ssc-miRNAs to seed them into a multiple sequence alignment tool, namely ClustlW⁵⁴. Here a pairwise sequence alignment between DE ssc-miRNAs and human reference genome assembly (version hg38) was conducted with specified loci position to meet the following parameters, namely the k-tuple word size 1 and window size 5 with top diagonals of 5 with percent method. Gap penalty of 10 and penalty of extension 0.1 with BLOSUM scoring weight matrix were applied. As a result, we identified a set of ssc-miRNAs, which were sequentially homologues to the known human miRNAs. We named this set as sequentially conserved (sc)-miRNA or sc-miRNA.

In parallel, we used the miRBase to align the quality filtered and adaptor-trimmed reads to the human genome assembly, hg38. Thereby, we followed the previously described analysis pipeline with one important difference, namely the miRDeep2 core algorithm in miRBase curated known hsa-miRNAs, not ssc-miRNA. The resultant hsa-miRNA matrix were quality filtered and the counts were normalized to generate per sample hsa-miRNA set. Differential expression analysis performed at each of the eight post-TBI time points found those hsa-miRNAs, which were significantly different from the baseline formed by the pool of three pre-TBI data, with a cutoff of moderate t-test p<0.05. We named this set of hsa-miRNA as functionally similar (fs)-miRNA (fs-miRNA).

Statistical Analysis

GeneSpring (Agilent Technologies, Inc., Santa Clara, CA) was used to find statistically significant markers, compute principal component (PC) analysis and clustering analysis. Hierarchical clustering was conducted using Similarity Measure: Squared Euclidean similarity measures and

Wards linkage rule. Prism (GraphPad, Inc. San Diego, CA) was used for data visualization. Ingenuity pathway analysis (Agilent, Inc.) was used for functional analysis.

Four months old male GMPs were exposed to lethal dose of TBI. The plasma samples were collected seven days (C7) and one day (C1) pre-TBI. First irradiated plasma samples were collected six hours after TBI and we called it 0d time point; subsequently plasma samples were drawn from the same three GMPs at 7 different post-TBI time-points, namely 1 day (1d), 3 day (3d), 7 day (7d), 10 day (10d), 14 day (14d), 17 day (17d) and 20 day (20d) post-TBI.

### Example 2: Longitudinal Expression Dynamics of microRNA (miRNA)

The principal component analysis (PCA) plot revealed the underlying relationship among the time resolved miRNA expressions (FIG. 1B). The PC1 and PC2 explained 29.3% and 11.2% of total variance in the global miRNA landscape. Taking cues from the distributions of the samples across PCA plot, the time points were assigned ad hoc into four phases. Early phase included 0d, 1d, 3d and 7d post-TBI time points, which were marked by circles in FIG. 1B. Comprising 10d and 14d post-TBI time points, the Mid phase was marked by rectangles in FIG. 1B. Finally, the Late and LR phases included 17d and 20d post-TBI time points, respectively.

Differential analysis of Early phase found 45 upregulated and 43 down-regulated ssc-miRNAs from the pre-TBI baseline. Likewise, there were 22 upregulated and 106 down-regulated, 11 upregulated and 113 down-regulated, and 26 upregulated and 24 down-regulated ssc-miRNAs in Mid, Late and LR-phase, respectively. All of these ssc-miRNAs showed significant perturbations in comparison to the pre-TBI baseline. Together, there were 212 ssc-miRNAs, which were DE in at least one of the four temporal phases.

### Example 3: Multiple Sequence Alignment Between Human and Minipig miRNA Assembly

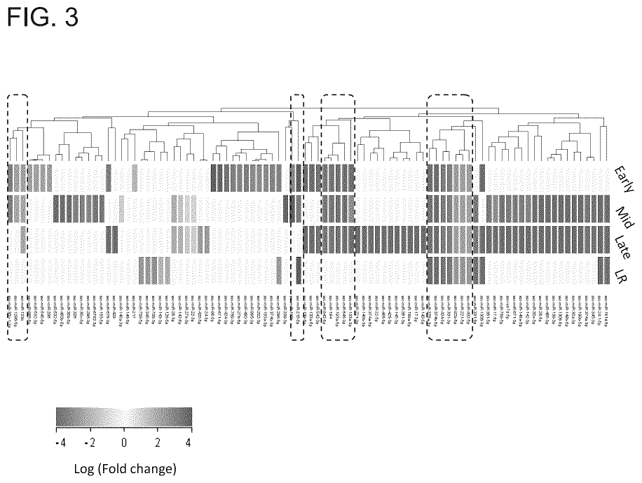

Objective of the analysis pipeline described in FIG. 1C was to determine a subset of these DE ssc-miRNAs that would have high translational potential. A multiple sequence alignments protocol⁵⁴ identified those hsa-miRNAs, which were sequentially conserved to 212 DE ssc-miRNA; and we named these 212 ssc-miRNA as sc-miRNA. In a parallel pipeline, the quality controlled NextSeq reads were directly blasted to human genome assembly and differential expression analysis was performed to curate 110 significantly different hsa-miRNAs from pre-TBI baseline. We labeled this second set as fs-miRNA. Ultimately, 212 sc-miRNAs and 110 fs-miRNAs were overlapped to find 92 miRNAs that were common between these two sets, and named as conserved-miRNA (cnvd-miRNA). In the hierarchical clustering of these cnvd-miRNAs (FIG. 3), four nodes were of particular interests, since they included those cnvd-miRNAs, which demonstrated longitudinally consistent trends of regulations (Table 1).

### Example 4: Longitudinal Profile of miRNA Enriched Networks

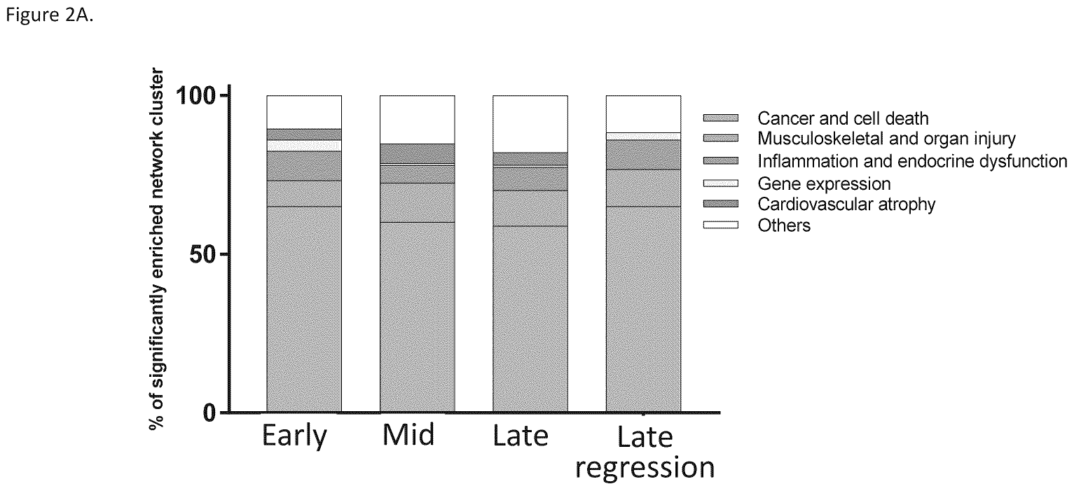

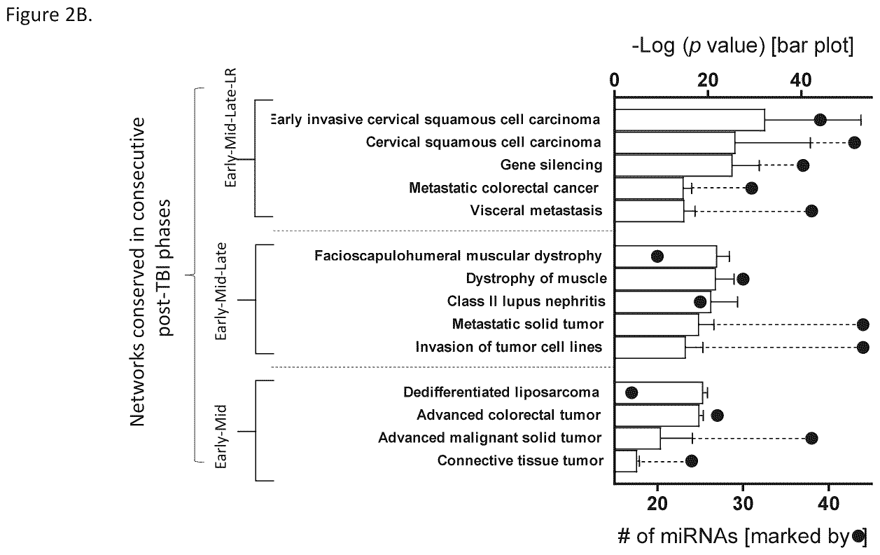

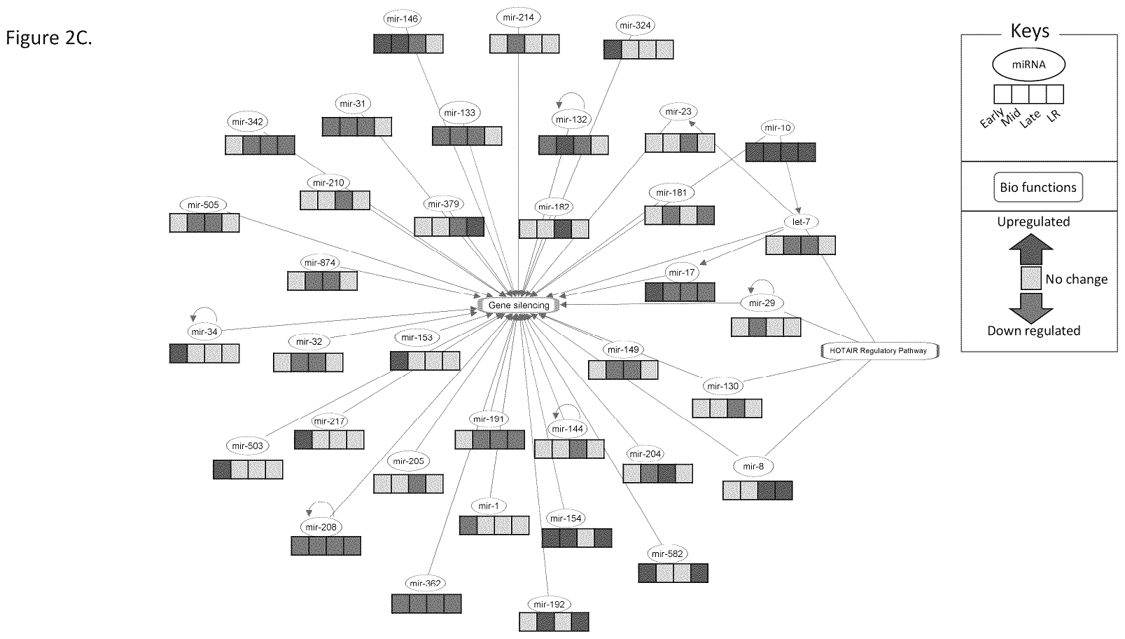

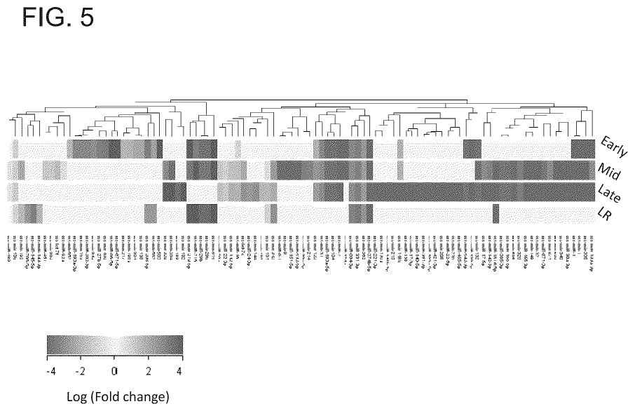

The entire list of 212 DE ssc-miRNAs enriched 206 networks, which were primarily sorted into six major categories, namely (i) cancer and cell death, (ii) musculoskeletal and organ injury, (iii) inflammation and endocrine dysfunction, (iv) gene expression, (v) cardiovascular atrophy and (vi) other; here the other set included cell movement, cell cycle, neurological illness, developmental disorder etc. A stacked bar chart (FIG. 2A) showed the longitudinal distribution of these network categories. The cancer related networks covered 60% of all networks across the timescale. There were 110 miRNAs enriching the cancer networks (FIG. 5), of which four miRNAs, namely ssc-miR-374b-5p, ssc-miR-331-3p, ssc-miR-664-3p, ssc-mir-362 were consistently down-regulated and one miRNA, namely ssc-mir-10b was consistently up-regulated across the timescale. Furthermore, ssc-miR-374b-5p and ssc-miR-331-3p were among the cnvd-miRNA set. In addition to cancer network, other dominating network categories included the musculoskeletal and organ injury, and inflammation and endocrine dysfunction, and these two categories covered 10.8% and 7.9% of all networks.

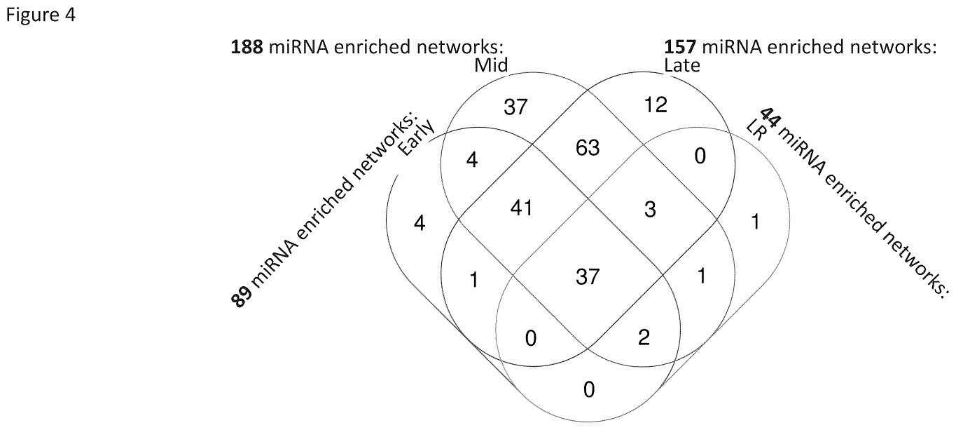

Overall, there were 86, 178, 151 and 43 networks, which were most significantly perturbed in the Early, Mid, Late and LR phases, respectively. A Venn diagram (FIG. 4) depicted the longitudinal distribution of these networks, as 39.8% of all of these networks remained perturbed starting from the Early phase. Ranked by the hypergeometric p-values, the most significantly perturbed networks were shown in FIG. 2B. Majority of these networks were linked to cancer. FIG. 5 showed a hierarchical clustering of cancer related 106 ssc-miRNAs.

The gene-silencing network, a candidate of the gene expression category was significantly perturbed across the entire post-TBI time scale (FIG. 2C). The gene-silencing network was enriched by 36 ssc-miRNAs, of which ssc-miR-362 and ssc-miR-208 were consistently down-regulated and ssc-miR-10 were consistently upregulated across the four post-TBI phases. Furthermore, there were two network candidates enlisted under musculoskeletal and organismal injury category, namely facioscapulohumeral muscular dystrophy and atrophy of muscle that emerged highly enriched across Early, Mid and Late phase. Muscular atrophy network was enriched by 36 miRNAs; notably, its two candidates, namely ssc-miR-374b-5p and miR-362 were consistently down regulated and ssc-miR-10 was consistently upregulated across the post-TBI phases.

### Discussion of Examples 1-5

Model Characteristics and Study Limitations

The prospect of circulating miRNAs as radiation biomarkers received additional traction by the growing number of studies that aimed to integrate miRNA expressions in blood serum or plasma. The major challenge of finding radiation marker is the near unavailability of ‘pure’ clinical samples without confounding factors, such as cancer. Therefore, the appeal of studying large animal models in the context of TBI is enormous. Further, it is essential to apply in silico mechanism to find markers of translational potential. We aim to bridge this knowledge gap by investigating a large animal model that was minipigs, which share many important traits with human. Next, analytical pipeline curated those markers, which were likely to be sequentially homologues with human and functionally similar in responding to lethal TBI. Present study is limited by a small sample size. Nevertheless, longitudinal analysis and multi-omics interrogation were undertaken to enhance statistical power. Sequencing of small RNA pool and multiple alignment of the reads ensued to find viable markers of translational potential.

Acute Impact of Total Body Irradiation was Severe

The immediate impact of lethal radiation was exhaustive to both miRNA profile in the minipig model, and this condition essentially mirrored a previously published TBI mouse model⁵³. Systematic differential expression analysis found that the largest portion of all DE miRNAs were perturbed at the Early phase. PCA plot of miRNA revealed an underlying longitudinal profile driven by host response to TBI. C1 and C2 samples were clustered with 0d, 1d, 3d and 7d; together these post-TBI time points were named as Early phase. Clustered separately from the Early phase in the PCA plot, two subsequent time points (10d and 14d post-TBI) were jointly named as Mid phase. Subsequent time point, 17d post-TBI diverged furthest from the pre-TBI, Early and Mid phases, respectively, and we binned 17d as the lone entrant of Late phase. Till the Late phase, the Euclidian divergence in PCA plot increased from the pre-TBI baseline in proportion to the times since irradiation, which mirrored the trend previously reported in mouse model⁵³. However, this trend was breached at 20d post-TBI in GMPs, as this very late time point clustered back to the Early phase. Hence, we named 20d time point as Late Regression (LR) phase. This incident potentially highlighted the differences between the mouse and minipig models. The proportionately larger animal model opened a scope to probe the effects of lethal radiation for a longer time window, which remained elusive to the mouse study.

Interestingly, the longitudinal regression trend of miRNA expression profile was not mirrored by the network analysis. Majority of the networks were triggered at the Early phase underscoring the need of immediate intervention to most effectively combat lethal radiation. The cancer and cell death related networks were consistently perturbed across the post-TBI time course. Decrement of musculoskeletal fitness, a typical signature of radiation was also triggered at the Early phase.

An in Silico Approach to Determine Cross-Species Conserved Markers

A rather cohesive perturbations of miRNA networks across different animal models^{53,59} essentially promoted these small molecules as putative radiation markers. However, there were multiple challenges to translate these candidates to clinically actionable items, since the heterogeneity between human and mouse genomic makeups are more pronounced in the noncoding transcriptional elements than their coding regions⁶⁰. Hence, we aimed to identify those miRNAs markers, which not only share high sequence homology between human and minipig, but also likely to present a cross-species conserved functional response to lethal radiation. Towards this objective, a multiple sequence alignment method curated the sequentially homologous miRNAs between minipig and human.

A parallel analysis curated those miRNAs, which could possibly exhibit functionally coherent response to TBI between human and minipig. In this in silico undertaking, the miRNA sequence reads were mapped to human miRNA assembly. Subsequent differential analysis mined a set of hsa-miRNAs, which were possibly captured a human-like TBI response. Using a Venn-diagram based approach, we curated 93 cnvd-miRNAs, which were common between these two aforementioned sets enlisting the sequentially conserved and functionally similar miRNAs between human and minipig, respectively. Supplementary data provided the citation history and relevant descriptions of these 92 cnvd-miRNAs. Briefly, this list was an assortment of novel predictive candidates and acknowledged markers of radiation and cancer. For instance, miR-24-1-5p, miR-30e-3p and miR-28-5p were extensively linked to minipig radiation models; in addition, a dominating subset of cnvd-miRNAs including miR-142-3p, miR-148a-5p, miR-455-3p, miR-139-3p, miR-1306-5p etc. were occasionally linked to minipig radiation models. In addition, there are some candidates such as miR-30a-3p and miR-199b-5p, which had not previously linked to minipig radiation model.

In conclusion, we presented a list of predictive TBI biomarkers with translational potential. This putative panel of biomarkers showed sequence homology with human and likely to respond to TBI in a phylogenetically coherent manner. An extensive citation search also imparted confidence about the relevance of several candidates to radiation. Systematic investigation of these markers would yield time sensitive miRNA panel capable of revealing radiation history and predicting disease onset.

All patent and literature references cited in the present specification are hereby incorporated by reference in their entirety.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order, quantity or importance, but rather the terms first, second, etc., are used to distinguish one element from another.

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

TABLE 1
A clinically actionable panel of microRNAs (miRNA)
markers of early response to lethal total body irradiation
(TBI). This miRNA panel met two selection criteria.
(i) this is a subset of 92 cnvd-miRNAs, which are
likely to be sequentially conserved and functionally
similar miRNAs between human and minipig. (iii) In sus
scrofa, these miRNAs were consistently expressed in at least two
consecutive time points starting from the Early post-TBI phase.

| | Log₂(fold change) | | | | |
|---|---|---|---|---|---|
| microRNA | Early | Mid | Late | LR | Biofunction |
| miR-432-5p* | −2.61 | −2.49 | −2.49 | −2.70 | Linked to |
| | | | | | carcinogenesis and |
| | | | | | cardiac atrophy |
| miR-221-5p* | −2.66 | −2.62 | −2.62 | −2.83 | Inhibited expression |
| | | | | | promotes cancer |
| | | | | | (PMID: 31788114) |
| miR-92b-5p* | −2.75 | −2.62 | −2.62 | −2.83 | Potential biomarker |
| | | | | | of heart failure |
| miR-331-3p* | −3.44 | −3.40 | −3.40 | −3.60 | Linked to |
| | | | | | carcinogenesis |
| miR-424-5p* | −3.54 | −3.49 | −3.49 | −3.70 | Associated with |
| | | | | | muscle wasting |
| miR-374b-5p* | −4.05 | −4.00 | −4.00 | −4.21 | Cancer, muscle |
| | | | | | atrophy |
| miR-376a-3p* | −3.92 | −3.80 | −3.80 | −4.00 | Linked to |
| | | | | | carcinogenesis |
| miR-133a-3p* | −4.44 | −3.10 | −9.61 | — | Linked to |
| | | | | | carcinogenesis |
| miR-545-3p* | −4.31 | −4.19 | −4.19 | — | Linked to cell |
| | | | | | migration, |
| | | | | | proliferation and |
| | | | | | invasion |
| miR-193a-5p | −3.84 | −3.80 | −3.80 | — | Linked to |
| | | | | | carcinogenesis |
| miR-184* | −3.80 | −3.76 | −3.76 | — | Linked to |
| | | | | | carcinogenesis |
| miR-542-5p | −3.79 | −3.75 | −3.75 | — | Inhibited expression |
| | | | | | promotes cancer |
| miR-133b | −2.57 | −2.44 | −2.44 | — | Inhibited expression |
| | | | | | promotes cancer |
| miR-218-5p* | 4.02 | 3.27 | — | 4.83 | Linked to |
| | | | | | carcinogenesis |
| miR-190b* | 3.01 | 6.27 | — | — | Promotes cancer |
| miR-1296-5p | −2.53 | −2.40 | — | — | Inhibited expression |
| | | | | | promotes cancer |
| miR-30c-1-3p | −3.54 | −3.49 | — | — | Inhibited expression |
| | | | | | promotes cancer |

*All of these miRNAs differentially expressed from the pre-TBI baseline with t-test p < 0.05; a subset of these miRNAs also met FDR cut-off 0.05, and those are marked with *.

### REFERENCES

1 Gale, R. P. & Armitage, J. O. Are We Prepared for Nuclear Terrorism? New England Journal of Medicine 378, 1246-1254, doi:10.1056/NEJMsr1714289 (2018).
2 Singh, V. K., Newman, V. L., Berg, A. N. & MacVittie, T. J. Animal models for acute radiation syndrome drug discovery. Expert Opin Drug Discov 10, 497-517, doi:10.1517/17460441.2015.1023290 (2015).
3 Gale, R. P., Armitage, J. O. & Hashmi, S. K. Emergency response to radiological and nuclear accidents and incidents. British Journal of Haematology n/a, doi:10.1111/bjh.16138.
4 DiCarlo, A. L. et al. Radiation injury after a nuclear detonation: medical consequences and the need for scarce resources allocation. Disaster Med Public Health Prep 5 Suppl 1, S32-44, doi:10.1001/dmp.2011.17 (2011).
5 Schaue, D. et al. Radiation and inflammation. Seminars in radiation oncology 25, 4-10, doi:10.1016/j.semradonc.2014.07.007 (2015).
6 Satyamitra, M. M., DiCarlo, A. L. & Taliaferro, L. Understanding the Pathophysiology and Challenges of Development of Medical Countermeasures for Radiation-Induced Vascular/Endothelial Cell Injuries: Report of a NIAID Workshop, Aug. 20, 2015. Radiation research 186, 99-111, doi:10.1667/RR14436.1 (2016).
7 Lorenz, E. & Congdon, C. C. Radioactivity; biologic effects of ionizing radiations. Annu Rev Med 5, 323-338, doi:10.1146/annurev.me.05.020154.001543 (1954).
8 Plett, P. A. et al. Establishing a murine model of the hematopoietic syndrome of the acute radiation syndrome. Health Phys 103, 343-355, doi:10.1097/HP.0b013e3182667309 (2012).
9 Williams, J. P. et al. Animal models for medical countermeasures to radiation exposure. Radiat Res 173, 557-578, doi:10.1667/rr1880.1 (2010).
10 Harding, J. D. Nonhuman Primates and Translational Research: Progress, Opportunities, and Challenges. ILAR Journal 58, 141-150, doi:10.1093/ilar/i1x033 (2017).
11 Verdier, J.-M. et al. Lessons from the analysis of nonhuman primates for understanding human aging and neurodegenerative diseases. Frontiers in Neuroscience 9, doi:10.3389/fnins.2015.00064 (2015).
12 Farese, A. M. et al. A nonhuman primate model of the hematopoietic acute radiation syndrome plus medical management. Health Phys 103, 367-382, doi:10.1097/HP.0b013e31825f75a7 (2012).
13 Kobayashi, E., Hishikawa, S., Teratani, T. & Lefor, A. T. The pig as a model for translational research: overview of porcine animal models at Jichi Medical University. Transplant Res 1, 8-8, doi:10.1186/2047-1440-1-8 (2012).
14 Nielsen, K. L. et al. Similar metabolic responses in pigs and humans to breads with different contents and compositions of dietary fibers: a metabolomics study. The American Journal of Clinical Nutrition 99, 941-949, doi:10.3945/ajcn.113.074724 (2014).
15 Matthan, N. R. et al. The Ossabaw Pig Is a Suitable Translational Model to Evaluate Dietary Patterns and Coronary Artery Disease Risk. J Nutr 148, 542-551, doi:10.1093/jn/nxy002 (2018).
16 Debus, E. S. et al. Feasibility Study of a Novel Thoraco-abdominal Aortic Hybrid Device (SPIDER-graft) in a Translational Pig Model. Eur J Vasc Endovasc Surg 55, 196-205, doi:10.1016/j.ejvs.2017.11.018 (2018).
17 Emmert, M. Y. et al. Safety and efficacy of cardiopoietic stem cells in the treatment of post-infarction left-ventricular dysfunction—From cardioprotection to functional repair in a translational pig infarction model. Biomaterials 122, 48-62, doi:10.1016/j.biomaterials.2016.11.029 (2017).
18 Gallo, M. et al. The Vietnamese pig as a translational animal model to evaluate tissue engineered heart valves: promising early experience. Int J Artif Organs 40, 142-149, doi:10.5301/ijao.5000568 (2017).
19 Kaiser, E. E. et al. Characterization of tissue and functional deficits in a clinically translational pig model of acute ischemic stroke. Brain Res 1736, 146778, doi:10.1016/j.brainres.2020.146778 (2020).
20 Kinder, H. A., Baker, E. W. & West, F. D. The pig as a preclinical traumatic brain injury model: current models, functional outcome measures, and translational detection strategies. Neural Regen Res 14, 413-424, doi:10.4103/1673-5374.245334 (2019).
21 Kobayashi, E., Hishikawa, S., Teratani, T. & Lefor, A. T. The pig as a model for translational research: overview of porcine animal models at Jichi Medical University. Transplant Res 1, 8, doi:10.1186/2047-1440-1-8 (2012).
22 Vasamsetti, B. M. K. et al. Validation of mouse phosphoprotein enriched in astrocyte 15 (mPEA15) expressing transgenic pig as a potential model in diabetes translational research. 3 Biotech 10, 34, doi:10.1007/s13205-019-2021-0 (2020).
23 McAnulty, P. A., Dayan, A. D., Ganderup, N.-C. & Hastings, K. L. The minipig in biomedical research. (CRC press, 2011).
24 Fang, X. et al. Genomic data from the Wuzhishan inbred pig (Sus scrofa). GigaScience (2012).
25 Moroni, M. et al. Hematopoietic radiation syndrome in the Gottingen minipig. Radiat Res 176, 89-101, doi:10.1667/rr2481.1 (2011).
26 Moroni, M. et al. Hematological changes as prognostic indicators of survival: similarities between Gottingen minipigs, humans, and other large animal models. PLoS One 6, e25210, doi:10.1371/journal.pone.0025210 (2011).
27 Kaur, A. et al. Development of a pediatric model of hematopoietic acute radiation syndrome (H-ARS) and countermeasure testing using the Gottingen minipigs. Rad Appl 2, 75-81 (2017).
28 Kenchegowda, D. et al. Selective Insulin-like Growth Factor Resistance Associated with Heart Hemorrhages and Poor Prognosis in a Novel Preclinical Model of the Hematopoietic Acute Radiation Syndrome. Radiat Res 190, 164-175, doi:10.1667/rr14993.1 (2018).
29 Moroni, M. et al. The Gottingen minipig is a model of the hematopoietic acute radiation syndrome: G-colony stimulating factor stimulates hematopoiesis and enhances survival from lethal total-body y-irradiation. Int J Radiat Oncol Biol Phys 86, 986-992, doi:10.1016/j.ijrobp.2013.04.041 (2013).
30 Legesse, B. et al. Neulasta Regimen for the Hematopoietic Acute Radiation Syndrome: Effects Beyond Neutrophil Recovery. International Journal of Radiation Oncology, Biology, Physics 103, 935-944, doi:10.1016/j.ijrobp.2018.11.043 (2019).
31 Chopra, S. et al. Gene Expression Profiles from Heart, Lung and Liver Samples of Total-Body-Irradiated Minipigs: Implications for Predicting Radiation-Induced Tissue Toxicity. Radiat Res 194, 411-430, doi:10.1667/rade-20-00123.1 (2020).
32 Malachowska, B. et al. Circulating microRNAs as Biomarkers of Radiation Exposure: A Systematic Review and Meta-Analysis. International Journal of Radiation Oncology, Biology, Physics 106, 390-402, doi:10.1016/j.ijrobp.2019.10.028 (2020).
33 Chakraborty, N. et al. microRNA and Metabolite Signatures Linked to Early Consequences of Lethal Radiation. Scientific Reports 10, 5424, doi:10.1038/s41598-020-62255-w (2020).
34 Singh, V. K. & Pollard, H. B. Ionizing radiation-induced altered microRNA expression as biomarkers for assessing acute radiation injury. Expert Rev Mol Diagn 17, 871-874, doi:10.1080/14737159.2017.1366316 (2017).
35 Ghosh, S. P. et al. Gamma-Tocotrienol Modulates Radiation-Induced MicroRNA Expression in Mouse Spleen. Radiat Res 185, 485-495, doi:10.1667/rr14248.1 (2016).
36 Acharya, S. S. et al. Serum microRNAs are early indicators of survival after radiation-induced hematopoietic injury. Sci Transl Med 7, 287ra269, doi:10.1126/scitranslmed.aaa6593 (2015).
37 Tomasik, B., Fendler, W. & Chowdhury, D. Serum microRNAs—potent biomarkers for radiation biodosimetry. Oncotarget 9, 14038-14039, doi:10.18632/oncotarget.24381 (2018).
38 Li, X. H. et al. Delta-tocotrienol suppresses radiation-induced microRNA-30 and protects mice and human CD34+ cells from radiation injury. PLoS One 10, e0122258, doi:10.1371/journal.pone.0122258 (2015).
39 Fendler, W. et al. Evolutionarily conserved serum microRNAs predict radiation-induced fatality in nonhuman primates. Sci Transl Med 9, doi:10.1126/scitranslmed.aa12408 (2017).
40 Menon, S. S. et al. Radiation Metabolomics: Current Status and Future Directions. Front Oncol 6, 20, doi:10.3389/fonc.2016.00020 (2016).
41 Ghosh, S. P. et al. Metabolomic changes in gastrointestinal tissues after whole body radiation in a murine model. Mol Biosyst 9, 723-731, doi:10.1039/c3mb25454b (2013).
42 Goudarzi, M. et al. The effect of low dose rate on metabolomic response to radiation in mice. Radiation and environmental biophysics 53, 645-657, doi:10.1007/s00411-014-0558-1 (2014).
43 Mak, T. D. et al. Exposure to ionizing radiation reveals global dose- and time-dependent changes in the urinary metabolome of rat. Metabolomics: Official journal of the Metabolomic Society 11, 1082-1094, doi:10.1007/s11306-014-0765-4 (2015).
44 Wang, C., Yang, J. & Nie, J. Plasma phospholipid metabolic profiling and biomarkers of rats following radiation exposure based on liquid chromatography-mass spectrometry technique. Biomedical chromatography: BMC 23, 1079-1085, doi:10.1002/bmc.1226 (2009).
45 Park, H. M. et al. Mass spectrometry-based metabolite profiling in the mouse liver following exposure to ultraviolet B radiation. PloS one 9, e109479, doi:10.1371/journal.pone.0109479 (2014).
46 Cheema, A. K. et al. Liver metabolomics reveals increased oxidative stress and fibrogenic potential in gfrp transgenic mice in response to ionizing radiation. J Proteome Res 13, 3065-3074, doi:10.1021/pr500278t (2014).
47 Boguszewicz, Ł. et al. 1H NMR based metabolomic approach to monitoring of the head and neck cancer treatment toxicity. Metabolomics: Official journal of the Metabolomic Society 12, 102, doi:10.1007/s11306-016-1043-4 (2016).
48 Ros-Mazurczyk, M. et al. Ionizing radiation affects profile of serum metabolites:
increased level of 3-hydroxybutyric acid in serum of cancer patients treated with radiotherapy. Acta biochimica Polonica 64, 189-193, doi:10.18388/abp.2016_1301 (2017).
49 Bacarella, N. et al. Whole Body Irradiation Induces Diabetes and Adipose Insulin Resistance in Nonhuman Primates. International Journal of Radiation Oncology, Biology, Physics 106, 878-886, doi:10.1016/j.ijrobp.2019.11.034 (2020).
50 Mazo, I. B., Quackenbush, E. J., Lowe, J. B. & von Andrian, U. H. Total body irradiation causes profound changes in endothelial traffic molecules for hematopoietic progenitor cell recruitment to bone marrow. Blood 99, 4182-4191, doi:10.1182/blood.v99.11.4182 (2002).
51 Hunnisett, A. G., Kars, A., Howard, J. M. H. & Davies, S. Changes in plasma amino acids during conditioning therapy prior to bone marrow transplantation: Their relevance to antioxidant status. Amino Acids 4, 177-185, doi:10.1007/BF00805813 (1993).
52 Byrum, S. D. et al. Time- and radiation-dose dependent changes in the plasma proteome after total body irradiation of non-human primates: Implications for biomarker selection. PLOS ONE 12, e0174771, doi:10.1371/journal.pone.0174771 (2017).
53 Chakraborty, N. et al. micro RNA and Metabolite Signatures Linked to Early Consequences of Lethal Radiation. J Scientific reports 10, 1-13 (2020).
54 Thompson, J. D., Gibson, T. J. & Higgins, D. G. Multiple sequence alignment using ClustalW and ClustalX. J Current protocols in bioinformatics, 2.3. 1-2.3. 22 (2003).
55 Chakraborty, N. et al. Gene-metabolite profile integration to understand the cause of spaceflight induced immunodeficiency. npj Microgravity 4, 4 (2018).
56 Chakraborty, N. et al. Gene-metabolite network linked to inhibited bioenergetics in association with spaceflight-induced loss of male mouse quadriceps muscle. Journal of Bone Mineral Research
   (2020).
57 Chavakis, T., Mitroulis, I. & Hajishengallis, G. J. N. i. Hematopoietic progenitor cells as integrative hubs for adaptation to and fine-tuning of inflammation. 20, 802-811 (2019).
58 Gurina, T. S. & Mohiuddin, S. S. in StatPearls [Internet] (StatPearls Publishing,
   2020).
59 Tomasik, B., Fendler, W. & Chowdhury, D. J. O. Serum microRNAs-potent biomarkers for radiation biodosimetry. 9, 14038 (2018).
60 Breschi, A., Gingeras, T. R. & Guigó, R. J. N. R. G. Comparative transcriptomics in human and mouse. 18, 425 (2017).

What is claimed is:

1. A method for detecting exposure to ionizing radiation in a subject, comprising:
assaying a biological sample to detect the presence of biomarkers that are downregulated or upregulated relative to a normal non-irradiated biological sample, wherein the biological sample is, optionally, a blood sample comprising serum or plasma, from a subject at risk of exposure to ionizing radiation to determine if radiation exposure has occurred, and, optionally, the approximate time lapsed since the radiation exposure;
wherein:
viii. downregulation of one or more Group A miRNAs relative to normal non-irradiated serum or plasma sample, wherein Group A miRNAs comprise miR-432-5p, miR-221-5p, miR-92b-5p, miR-331-3p, miR-424-5p, miR-376a-3p and/or miR-374b-5p; and/or
ix. upregulation of one or more Group B1 miRNAsrelative to normal non-irradiated serum or plasma sample, wherein Group B1 miRNAs miR-218-5p, miR-96-5p, miR-671-5p, miR-424-3p, miR-490-3p, miR-296-5p, miR-769-3p, miR-27b-5p, miR-190b, miR-374b-3p, miR-193a-3p, miR-202-3p, miR-885-3p and/or miR-217; and/or
x. downregulation of Group B2 miRNAs relative to normal non-irradiated serum or plasma sample, wherein Group B2 miRNAs comprise miR-532-3p, miR-885-5p, miR-545-5p, miR-1296-5p, miR-133b, miR-206, miR-432-5p, miR-221-5p, miR-92b-5p, miR-331-3p, miR-424-5p, miR-30c-1-3p, miR-676-3p, miR-542-3p, miR-542-5p, miR-184, miR-193a-5p, miR-376a-3p, miR-374b-5p, miR-139-5p, miR-545-3p, miR-133a-3p, miR-133a-5p and/or miR-1306-3p; and/or
xi. upregulation of one or more Group C1 miRNAsrelative to normal non-irradiated serum or plasma sample, wherein Group C1 miRNAs comprise, miR-218-5p, miR-190b, miR-339-3p, miR-429 and/or miR-676-3p; and/or
xii. downregulation of one or more Group C2 miRNAs relative to normal non-irradiated serum or plasma sample, wherein Group C2 miRNAs comprise let-7d-5p, miR-1306-5p, miR-139-3p, miR-140-5p, miR-142-3p, miR-142-5p, miR-145-3p, miR-148a-5p, miR-148a-5p, miR-148b-5p, miR-155-5p, miR-17-3p, miR-17-5p, miR-181d-5p, miR-199a-5p, miR-199b-5p, miR-221-3p, miR-22-3p, miR-22-5p, miR-24-1-5p, miR-24-3p, miR-27b-3p, miR-28-3p, miR-28-5p, miR-296-3p, miR-30a-3p, miR-30b-5p, miR-30c-5p, miR-30e-3p, miR-326, miR-331-5p, miR-345-3p, miR-361-3p, miR-361-5p, miR-374a-3p, miR-374a-5p, miR-425-3p, miR-425-5p, mir-450b-5p, miR-455-3p, miR-455-5p, miR-532-5p, miR-671-3p, miR-6782-3p, miR-769-5p, miR-1296-5p, miR-133b, miR-542-5p, miR-184, miR-193a-5p, miR-545-3p, miR-133a-3p, miR-432-5p, miR-221-5p, miR-92b-5p, miR-331-3p, miR-424-5p, miR-30c-1-3p, miR-542-3p, miR-376a-3p, miR-374b-5p, miR-139-5p, miR-545-3p, miR-133a-3p, miR-133a-5p and/or miR-1306-3p; and/or
xiii. upregulation of one or more Group D1 miRNAsrelative to normal non-irradiated serum or plasma sample, wherein Group D1 miRNAs comprise miR-218-5p, miR-296-5p, miR-126-5p, miR-424-3p, miR-143-3p, miR-345-3p, miR-708-3p and/or miR-708-5p; and/or
xiv. downregulation of one or more Group D2 miRNAsrelative to normal non-irradiated serum or plasma sample, wherein Group D2 miRNAs comprise miR-181d-5p, miR-24-1-5p, miR-331-5p, miR-432-5p, miR-221-5p, miR-92b-5p, miR-331-3p, miR-424-5p, miR-376a-3p, miR-374b-5p and/or miR-1306-3p,
are conditions indicative of radiation exposure in the subject, and,
optionally, administering a radiation injury therapy to the subject if any of conditions i-vii are present.

2. The method of claim 1, wherein upregulation of Group B1 miRNA's or downregulation of Group B2 miRNA's, or a combination thereof, is an early marker indicative of approximately 0-11 months post-exposure to ionizing radiation.

3. The method of claim 1, wherein upregulation of Group C1 miRNA's or downregulation of Group C2 miRNA's, or a combination thereof, is an intermediate marker indicative of approximately 5-9 months post-exposure to ionizing radiation.

4. The method of claim 1, wherein upregulation of Group D1 miRNA's or downregulation of Group D2 miRNA's, or a combination thereof, is a late marker indicative of approximately at least 12 months post-exposure to ionizing radiation.

5. A radiation exposure detection kit, comprising:
one or more reagents for detecting one or more biomarkers, comprising:
a first time-independent marker for detecting one or more of the following miRNAs in a blood serum or plasma sample, comprising: miR-432-5p, miR-221-5p, miR-92b-5p, miR-331-3p, miR-424-5p, miR-376a-3p and miR-374b-5p;
a second, early detection biomarker for detecting upregulation of one or more of Group B1 miRNA's or downregulation of one or more of Group B2 miRNA's, or a combination thereof;
a third, intermediate detection biomarker for detecting upregulation of one or more of Group C1 miRNA's or downregulation of one or more of Group C2 miRNA's, or a combination thereof; and/or
a fourth, late detection biomarker for detecting upregulation of one or more of Group D1 miRNA's or downregulation of one or more of Group D2 miRNA's, or a combination thereof;
wherein said kit is configured to detect a) if exposure to ionizing radiation has occurred, and b) the approximate time since exposure.

6. The kit of claim 5, wherein the one or more reagents comprises primers directed to the one or more biomarkers.

7. The kit of claim 5, wherein the one or more reagents comprises probes directed to the one or more biomarkers.

8. The kit of claim 7, wherein the probes comprise a detectable label.

9. A method of determining biomarkers indicative of radiation exposure, the method comprising
a) irradiating a test animal, to produce an irradiated animal;
b) collecting a test sample from the irradiated animal;
c) analyzing the test sample for differentially expressed miRNAs relative to a control animal; and
d) identifying differentially expressed miRNAs that are mapped to human miRNAs.

10. The method of claim 9, wherein identifying comprises identifying differentially expressed miRNAs in the test animal that are conserved with respect to human miRNAs or identifying differentially expressed miRNAs functionally similar to human miRNAs.

11. The method of claim 10, wherein identifying conserved with respect to human miRNAs comprises
pooling differentially expressed miRNAs,
subjecting pooled differentially expressed miRNAs to a multiple sequence alignment tool, and
conducting pairwise sequence alignment between test animal miRNA and human genome at predetermined loci position.

12. The method of claim 11, wherein the multiple sequence alignment tool comprises CLUSTL4, and conducting comprises applying parameters for alignment comprising a k-tuple word size 1 and window size 5 with top diagonals of 5 with percent method, and a gap penalty of 10 and penalty of extension 0.1 with BLOSUM scoring weight matrix.

13. The method of claim 10, wherein identifying differentially expressed miRNAs functionally similar to human miRNAs comprises
pooling differentially expressed miRNAs,
subjecting pooled differentially expressed miRNAs to a multiple sequence alignment tool, and
conducting pairwise sequence alignment between test animal miRNA and human genome at predetermined loci position, wherein conducting pairwise sequence alignment comprises subjecting differentially expressed miRNAs to miRDeep2 core algorithm in miRbase.

14. A method for detecting exposure to ionizing radiation in a subject, comprising:
Using the kit of claim 5 to assay a biological sample for the presence of biomarkers that are downregulated or upregulated relative to a normal non-irradiated biological sample, wherein the biological sample is, optionally, a blood sample comprising serum or plasma, from a subject at risk of exposure to ionizing radiation to determine if radiation exposure has occurred, and, optionally, the approximate time lapsed since the radiation exposure.

15. The method of claim 14, wherein the kit comprises wherein the one or more reagents of the kit comprise primers directed to the one or more biomarker and probes directed to the one or more biomarkers.

16. The method of claim 15, wherein the method comprises subjecting the biological sample to an amplification reaction.

17. The method of claim 16, wherein the amplification reaction comprises Polymerase Chain Reaction (PCR), Real-Time Polymerase Chain Reaction (RT-PCR), Quantitative Polymerase Chain Reaction (qPCR), or rolling circle amplification.

18. The method of claim 1, wherein detecting comprises subjecting the biological sample to an amplification reaction using primers directed to the one or more biomarkers.

19. The method of claim 1, wherein detecting comprises subjecting the biological sample to hybridization-based methods, NanoString™ analysis, Northern Blot analysis, branched DNA (bDNA) signal amplification, in situ hybridization, sequencing-based methods, ribonuclease protection assay (RPA) and/or mass spectroscopy.

20. The method of claim 1, wherein the method comprises administering a radiation injury therapy to the subject.
